(19) United States
(12) Patent Application Publication
(10) Pub. No.: US 2017/0182268 A1
(43) Pub. Date: Jun. 29, 2017

(54) ASSEMBLY FOR A COUNTER MECHANISM FOR A DRUG DELIVERY DEVICE AND DRUG DELIVERY DEVICE

(71) Applicant: SANOFI SA, Vernier (CH)

(72) Inventor: Stefan MAYER, Frankfurt am Main (DE)

(21) Appl. No.: 15/313,140

(22) PCT Filed: May 18, 2015
(86) PCT No.: PCT/EP2015/060897

§ 371 Date: Nov. 22, 2016

(30) Foreign Application Priority Data
May 28, 2014 (EP) 14170382.7

(51) Int. Cl. A61M 15/00; G06M 1/24

(52) U.S. Cl. CPC A61M 15/0068; A61M 15/0073; G06M 1/24; A61M 15/009; G06M 1/041

(57) ABSTRACT

An assembly for a counter mechanism for a drug delivery device is described comprising a drive member, wherein the drive member is adapted and arranged to be rotated in a first rotational direction and to be prevented from rotation in a second rotational direction which direction is opposite to the first rotational direction, an advancing member adapted and arranged to be axially moved and rotated with respect to the drive member, wherein the advancing member is adapted and arranged to mechanically cooperate with the drive member such that the drive member is rotated in the first rotational direction, and a display member which is configured to count a number of doses and to display the counted number of doses, wherein the assembly is adapted and arranged such that rotation of the drive member in the first rotational direction is converted into a counting movement of the display member. Furthermore, a drug delivery device comprising the assembly is described.

### CROSS REFERENCE TO RELATED APPLICATIONS

The present application is a U.S. National Phase Application pursuant to 35 U.S.C. §371 of International Application No. PCT/EP2015/060897 filed May 18, 2015, which claims priority to European Patent Application No. 14170382.7 filed May 28, 2014. The entire disclosure contents of these applications are herewith incorporated by reference into the present application.

### BACKGROUND

This disclosure relates to an assembly for a counter mechanism for a drug delivery device. Furthermore, the disclosure relates to a drug delivery device comprising the assembly. The drug delivery device may be an inhalator, in particular a dry powder inhaler. An inhalation device is usually activated by a user's suction airstream and is intended for the inhalation of a substance, in particular a powdery substance. An inhalation device is described in document WO 2009/065707 A1, for example.

However, the assembly may also be suitable for other drug delivery devices, e.g. an injector such as an injection pen. In particular, the assembly may be used in a fixed-dose drug delivery device, i.e. a device in which the size of the drug which is delivered may not be varied by the user. Rather, in a fixed-dose device the size of the dose is set by the design of a dispensing mechanism.

### SUMMARY

It is an object of the present disclosure to provide an improved drug delivery device.

This object may, inter alia, be achieved by the subject-matter of the independent claim. Advantageous embodiments and refinements are the subject-matter of the dependent claims. However, further advantageous concepts may be disclosed herein besides the ones which are claimed.

One aspect of the present disclosure relates to an assembly. The assembly may be suitable to be used in a counter mechanism for a drug delivery device. The device may be an inhalation device, in particular a dry powder inhalator. The device may be adapted for dispensing a plurality of doses of a medication or substance.

The term “medication” or “substance”, as used herein may mean a pharmaceutical formulation containing at least one pharmaceutically active compound, for example for the treatment of obstructive airway or lung diseases such as asthma or chronic obstructive pulmonary disease (COPD), local respiratory tract oedema, inflammation, viral, bacterial, mycotic or other infection, allergies, diabetes mellitus.

The active pharmaceutical compound is preferably selected from the group consisting of active pharmaceutical compounds suitable for inhalation, preferably antiallergenic, antihistamine, anti-inflammatory, antitussive agents, bronchodilators, anticholinergic drugs, and combinations thereof.

The active pharmaceutical compound may for example be chosen from:

an insulin such as human insulin, e.g. a recombinant human insulin, or a human insulin analogue or derivative, a glucagon-like peptide (GLP-1) or an analogue or derivative thereof, or exendin-3 or exendin-4 or an analogue or derivative of exendin-3 or exendin-4;

an adrenergic agent such as a short acting β2-agonists (e.g. Salbutamol, Albuterol, Levosalbutamol, Fenoterol, Terbutaline, Pirbuterol, Procaterol, Bitolterol, Rimiterol, Carbuterol, Tulobuterol, Reproterol), a long acting β2-agonist (LABA, e.g. Arformoterol, Bambuterol, Clenbuterol, Formoterol, Salmeterol), an ultra LABA (e.g. Indacaterol) or another adrenergic agent (e.g. Epinephrine, Hexoprenaline, Isoprenaline (Isoproterenol), Orciprenaline (Metaproterenol));

a glucocorticoid (e.g. Beclometasone, Budesonide, Ciclesonide, Fluticasone, Mometasone, Flunisolide, Betamethasone, Triamcinolone);

an anticholinergic agent or muscarinic antagonist (e.g. Ipratropium bromide, Oxitropium bromide, Tiotropium bromide);

a mast cell stabilizer (e.g. Cromoglicate, Nedocromil);

a xanthine derivative (e.g. Doxofylline, Enprofylline, Theobromine, Theophylline, Aminophylline, Choline theophyllinate);

an eicosanoid inhibitor, such as a leukotriene antagonist (e.g. Montelukast, Pranlukast, Zafirlukast), a lipoxygenase inhibitor (e.g. Zileuton) or a thromboxane receptor antagonist (e.g. Ramatroban, Seratrodast);

a phosphodiesterase type-4 inhibitor (e.g. Roflumilast);

an antihistamine (e.g. Loratadine, Desloratadine, Cetirizen, Levocetirizine, Fexofenadine);

an allergen immunotherapy (e.g. Omalizumab);

a mucolytic (e.g. Carbocisteine, Erdosteine, Mecysteine);

an antibiotic or antimycotic;

or a combination of any two, three or more of the above-mentioned compound classes or compounds (e.g. Budesonide/Formoterol, Fluticasone/Salmeterol, Ipratropium bromide/Salbutamol, Mometasone/Formoterol);

or a pharmaceutically acceptable salt or solvate or esters of any of the above named compounds.

Pharmaceutically acceptable salts are for example acid addition salts and basic salts. Acid addition salts are e.g. a chloride, bromide, iodide, nitrate, carbonate, sulfate, methylsulfate, phosphate, acetate, benzoate, benzenesulfonate, fumarate, malonate, tartrate, succinate, citrate, lactate, gluconate, glutamate, edetate, mesylate, pamoate, pantothenate or a hydroxy-naphthoate salt. Basic salts are for example salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology. Pharmaceutically acceptable ester may for example be acetates, propionates, phosphates, succinates or etabonates.

Pharmaceutically acceptable solvates are for example hydrates.

The assembly may comprise a drive member. The drive member may be adapted and arranged to be rotated in a first rotational direction. The drive member may be rotatable about an axis, in particular a longitudinal axis of the assembly or of the device. The drive member may be prevented from rotation in a second rotational direction which direction is opposite to the first rotational direction. The first rotational direction may be the clockwise direction and the second rotational direction may be the anti-clockwise direction, for example. The drive member may be prevented from axial movement. The assembly may further comprise an advancing member. The advancing member may be adapted and arranged to be axially moved with respect to the drive member. The advancing member may be axially moveable in a first axial direction. The first axial direction may be a direction away from a dispensing end of the device. The advancing member may be moveable in a second axial direction which is opposite to the first axial direction.

Furthermore, the advancing member may be adapted and arranged to be rotated with respect to the drive member. The advancing member may be rotatable about an axis, in particular a longitudinal axis of the assembly or of the device. The advancing member may be configured to be rotated around the same rotational axis as the drive member. The advancing member may be rotatable in the second rotational direction with respect to the drive member. However, the advancing member may be configured to be prevented from rotation in the first rotational direction with respect to the drive member. Alternatively, rotation of the advancing member in the first rotational direction may be enabled and rotation in the second rotational direction with respect to the drive member may be prevented.

The advancing member may be adapted and arranged to mechanically cooperate with the drive member such that the drive member is rotated in the first rotational direction. The advancing member may mechanically cooperate with the drive member when the advancing member is axially moved, preferably axially moved in the first axial direction, with respect to the drive member. Upon mechanical cooperation of the advancing member and the drive member, the advancing member may advance the drive member in the first rotational direction. The advancing member may be prevented from rotation when the advancing member and the drive member mechanically cooperate. In this way, a reliable propulsion of the drive member is achieved. Additional components for driving the drive member, e.g. spring or resilient components, are redundant. Hence, a reliable and cost-effective device is achieved.

The assembly may further comprise a display member. The display member may be configured to count a number of doses and/or to display the counted number of doses. In particular, the display member may be configured to count and display the number of doses that have been delivered by the drug delivery device. Alternatively, the display member may be configured to count and to display the number of doses that are left in the drug delivery device before the drug delivery device is considered empty.

The display member may be configured to be moved by the rotation of the drive member in the first rotational direction. The display member may be moveable in a direction transverse to the rotational axis of the drive member. In particular, the assembly may be adapted and arranged such that rotation of the drive member in the first rotational direction is converted into a counting movement of the display. Accordingly, movement, in particular axial movement, of the advancing member is converted into the rotational movement of the drive member for driving the display member. In this way, provision of a very stable counter mechanism and, hence, a reliable drug delivery device is facilitated.

Due to the conversion of the rotational movement of the drive member into the counting movement of the display member, a succeeding number of doses which can still be dispensed or which have already been dispensed may be displayed by the display member upon rotation of the drive member. In this way, provision of an effective and reliable counter mechanism and hence, of an improved device, is facilitated.

Further, the advancing member may comprise a first interaction member and the drive member may comprise a second interaction member, wherein the interaction members may be configured to mechanically cooperate with one another when they are arranged offset with respect to one another in a predetermined axial and/or angular orientation and when the advancing member is moved in the first axial direction such that the first interaction member slides along the second interaction member, thereby exerting a force onto the drive member for rotating the drive member in the first rotational direction, and wherein the interaction members may be configured to mechanically cooperate with one another when they are not arranged offset with respect to one another in the axial and/or angular orientation and when the advancing member is moved in the first axial direction such that the first interaction member is received in the second interaction member, without exerting a force onto the drive member.

According to one embodiment, the advancing member is configured to mechanically cooperate with the drive member when the advancing member is moved in a first axial direction with respect to the drive member. The advancing member and the drive member may be brought into engagement when the advancing member is moved in the first axial direction. The advancing member may be prevented from mechanical cooperation with the drive member when the advancing member is moved in a second axial direction with respect to the drive member. The advancing member and the drive member may be brought out of engagement when the advancing member is moved in the second axial direction. The second axial direction may be a dispensing or delivery direction, i.e. a direction in which the advancing member is moved during delivery of a dose of the substance. The second axial direction may be opposite to the first axial direction.

According to one embodiment, the assembly further comprises a plunger member. The plunger member is axially moveable. The plunger member may be adapted and arranged to be moved, in particular axially moved, during at least one of a dose dispensing operation and a dose setting operation. The plunger member may be moveable in the second axial direction during dose dispensing, for example. The plunger member may be moveable in the first axial direction during dose setting, for example. The plunger member may be configured to mechanically cooperate with the advancing member such that the advancing member is axially moveable with respect to the drive member. The plunger member and the advancing member may be engaged, preferably permanently engaged, with one another. The advancing member may be snap-fitted to the plunger member. In this way, movement of the plunger member may be effectively transferred into axial movement of the advancing member. The drive member may be driven due to movement, in particular axial movement, of the advancing member and, thus, of the plunger member.

The plunger member may comprise a first connection member, e.g. at least one protrusion. The advancing member may comprise a second connection member, e.g. a recess or indentation. The connection members are configured to mechanically cooperate with one another such that the plunger member and the advancing member engage one another. Further, the connection members are configured to mechanically cooperate with one another such that the plunger member is slideably connected to the advancing member. In other words, the plunger member may at least partly be moveable with respect to the advancing member. The plunger member may be axially moveable with respect to the advancing member for a predetermined distance. The distance may be determined by an axial extension of the second connection member, for example. Due to the slideable connection, the plunger member may be axially moveable with respect to the advancing member between a first axial position and a second axial position.

When the plunger member is arranged in the first axial position, movement of the plunger member in the first axial direction may be converted into movement of the advancing member in the first axial direction due to mechanical cooperation of the connection members. Accordingly, when the plunger member is arranged in the first axial position, relative movement of the plunger member and the advancing member may be prevented. When the advancing member is moved in the first axial direction, movement of the advancing member and, thus, movement of the plunger member, is converted into rotational movement of the drive member for driving the display member. Accordingly, the axial movement of the plunger member is transferred into the rotational movement of the drive member by means of the advancing member. In this way, provision of a very stable counter mechanism and, hence, a reliable drug delivery device is facilitated.

When the plunger member is arranged in the second axial position, movement of the plunger member in the second axial direction may be converted into movement of the advancing member in the second axial direction due to mechanical cooperation of the connection members. Accordingly, when the plunger member is arranged in the second axial position, relative movement of the plunger member and the advancing member may be prevented. When the plunger member is arranged in the second axial position, a dose may be delivered from the device. Accordingly, movement of the advancing member may occur only when a dose is delivered to a user. Unintentional movement of the advancing member and, thus, movement of the display member for displaying a succeeding number of doses may thus, be effectively prevented. In this way, provision of a safe and user-friendly device is facilitated.

When the plunger member is moved between the first axial position and the second axial position, movement of the advancing member with respect to the drive member is prevented due to the sliding engagement of the connection members. The plunger member may be moved from the first axial position towards the second axial position for initiating a dose delivery operation. The plunger member may be moved from the second axial position towards the first axial position for initiating a dose setting operation. When the plunger member is moved between the first axial position and the second axial position, the plunger member may be moved relative to the advancing member due to the slideable connection.

According to one embodiment, the drive member comprises a reverse rotation prevention feature. The reverse rotation prevention feature may be adapted and arranged to permit a rotation of the drive member in the first rotational direction. The reverse rotation prevention feature may be adapted and arranged to prevent a rotation of the drive member in the second rotational direction. The reverse rotation prevention feature may comprise a toothing. The toothing may be arranged on an outer surface of the drive member. The reverse rotation prevention feature may be configured to mechanically cooperate with a mating feature, e.g. a pawl, of the assembly. In this way, rotation of the drive member in the second rotational direction may be effectively prevented. Malfunction of the counter mechanism, e.g. a reverse counting, may thus be effectively prevented.

The advancing member may also comprise a reverse rotation prevention feature. The reverse rotation prevention feature may be adapted and arranged to permit a rotation of the advancing member in the second rotational direction, for example. The reverse rotation prevention feature may be adapted and arranged to prevent a rotation of the advancing member in the first rotational direction, for example. The reverse rotation prevention feature may comprise a toothing. The reverse rotation prevention feature may be configured to mechanically cooperate with a mating feature, e.g. a pawl, of the assembly. In this way, rotation of the advancing member in the first rotational direction may be effectively prevented.

According to one embodiment, the drive member comprises an opening. The drive member may be designed as a drum. The opening may be adapted and arranged to receive the advancing member. The advancing member may be at least partly introduced in the drive member. The advancing member may be slideably arranged within the drive member. In this way, provision of a compact device is facilitated.

According to one embodiment, the advancing member comprises a first interaction member. The first interaction member may comprise a set of teeth. The drive member may comprise a second interaction member. The second interaction member may comprise a set of mating teeth. The interaction members may be configured to mechanically cooperate with one another when the advancing member is moved in the first axial direction.

The interaction members may be arranged offset, in particular axially and/or angularly offset, with respect to one another when the interaction members start to mechanically cooperate. This means that a tip of a respective tooth of the first interaction member and a tip of a respective tooth of the second interaction member may have an axial and/or azimuthal distance with respect to one another before the interaction members mechanically cooperate. The tip of the respective tooth of the first interaction member may be arranged angularly offset from the tip of a respective tooth of the second interaction member for less than 1 mm, e.g. for 0.5 mm, 0.25 mm, 0.2 mm or 0.1 mm. The tip of the respective tooth of the first interaction member may be arranged axially offset from the tip of a respective tooth of the second interaction member for less than 0.5 mm, e.g. for 0.25 mm, 0.2 mm, 0.11 mm or 0.1 mm.

The offset between the tips of the teeth of the first interaction member and the second interaction member may depend on a height or axial extension of the teeth and/or on a distance between the teeth of the respective interaction member and/or on a slope of the teeth of the respective interaction member. The offset may be chosen such that, when the advancing member and the drive member are arranged in an end position with respect to one another after axial movement of the advancing member in the first axial direction was performed, the interaction members are in a meshed engagement. In other words, when the advancing member was completely moved into the first axial direction, the interaction members may complete engage one another.

The interaction members may be configured to slide along one another when the advancing member is moved in the first axial direction. Accordingly, the advancing member may exert a force onto the drive member for rotating the drive member in the first rotational direction when the advancing member is moved in the first axial direction. When the advancing member is arranged in the—axial—end position with respect to the drive member, the interaction members may be in the meshed engagement with one another. In other words, one tooth of the first interaction member may be positioned in a gap between two succeeding teeth of the second interaction member and vice versa. When the advancing member is arranged in the end position with respect to the drive member, the advancing member thus, can no longer exert the force onto the drive member.

According to one embodiment, the assembly further comprises a centering feature. The centering feature may comprise a prong or protrusion. The centering feature may comprise a toothing. The centering feature may be provided by the plunger member. In particular, the plunger member may comprise the centering feature. The plunger member and the centering feature may be integrally formed. The centering feature may be directed towards the first axial direction.

The advancing member comprises an interaction feature. The interaction feature may comprise a toothing. A graduation of the interaction feature may be smaller than a graduation of the first interaction member. In other words, the teeth of the interaction feature may be smaller and/or may have a smaller distance to one another as compared to the teeth of the first interaction member.

The interaction feature may be adapted and arranged to prevent rotation of the advancing member with respect to the drive member when the advancing member is moved in the first axial direction due to mechanical cooperation with the centering feature. Due to mechanical cooperation of the centering feature and the advancing member, rotation of the advancing member may be effectively prevented when the advancing member is moved towards the drive member. In this way, the previously described offset orientation of the interaction members is ensured before the interaction members start to mechanically cooperate.

According to one embodiment, the assembly further comprises a reset feature. The reset feature may comprise a prong or protrusion. The reset feature may be directed towards the first axial direction, for example. The reset feature may comprise a toothing. The advancing member may comprise a projection feature. The projection feature may comprise a toothing. A graduation of the projection feature, in particular of the toothing, may be smaller than a graduation of the first interaction member. In other words, the teeth of the projection feature may be smaller and/or may have a smaller distance to one another as compared to the teeth of the first interaction member. The projection feature may be arranged on an outer surface of the advancing member. The projection feature may protrude from the advancing member in the radial outward direction.

The reset feature and the projection feature may be configured to mechanically cooperate with one another when the advancing member is moved in the second axial direction. In an alternative embodiment, the reset feature and the projection feature may be configured to mechanically cooperate with one another when the advancing member is moved in the first axial direction. The reset feature and the projection feature may mechanically cooperate such that they slide along each other. Thereby, the reset feature may exert a force onto the advancing member for rotating the advancing member with respect to the drive member. Preferably, the advancing member is rotated in the second rotational direction. Alternatively, the advancing member may be rotated in the first rotational direction. The projection feature may also act as reverse rotation prevention feature of the advancing member for preventing a rotation of the advancing member in the first rotational direction due to mechanical cooperation with a pawl.

The reset feature and the projection feature may be arranged offset, in particular axially and/or angularly offset, with respect to one another before they start to mechanically cooperate. The reset feature and the projection feature may be arranged offset with respect to one another when the reset feature and the projection feature are arranged in an axial end position with respect to one other after the advancing member was completely moved in the second axial direction. In other words, there may always be an offset between the reset feature and the projection feature. In other words, a tip of a respective tooth of the projection feature and a tip of the reset feature are offset with respect to one another throughout the operation of the mechanism. In this way, rotation of the advancing member with respect to the drive member for arranging the interaction members offset from one another before the interaction members start to engage when the advancing member is moved in the first axial direction may be enabled throughout the operation of the device.

According to one embodiment, the display member and the drive member are adapted and arranged to mechanically cooperate with one another. In particular, the display member and the drive member may be in direct mechanical contact with one another. The display member may be attached to the drive member. The display member and the drive member may directly mechanically contact one another such that rotation of the drive member in the first rotational direction is converted into the counting movement of the display member. In this way, a reliable propulsion of the display member and, hence, an effective device is achieved.

According to one embodiment, the display member is at least partly wound around the drive member. In particular, as seen in plan view onto the display member, the display member may at least partly cover an outer surface of the drive member. The display member may be further wound onto the drive member upon rotation of the drive member in the first rotational direction. The display member may comprise a tape. The tape may comprise indicia configured to display information regarding the drug delivery device. The information may relate to a number of doses already dispensed. Alternatively, the information may relate to a number of doses which can still be dispensed. Accordingly, a user may realize at one glance how much doses have already been dispensed or how many doses still can be dispensed. In this way, provision of a safe and user-friendly device can be facilitated.

According to one embodiment, the drive member comprises at least one drive feature, preferably a plurality of drive features. The drive feature may comprise at least one protrusion. The display member may comprise at least one interaction member, preferably a plurality of interaction members. The interaction member may comprise at least one indentation or recess. The number of drive features may correspond to the number of interaction members. The drive feature may be adapted and arranged to mechanically cooperate with the interaction member of the display member. The interaction member and the drive feature may mechanically cooperate such that rotation of the drive member is converted into movement of the display member. In this way, an effective counter mechanism may be provided.

According to one embodiment, the assembly further comprises a rotating member. The rotating member may comprise a roll-up wheel. The rotating member may be adapted and arranged to be rotated in the first rotational direction. The rotating member may be rotatable about an axis, in particular a longitudinal axis of the assembly or of the device. The rotating member may be prevented from rotation in the second rotational direction. The rotating member may be prevented from axial movement. The rotating member may be coupled with the display member. The display member may be attached to, preferably non-releasably attached to, the rotating member. The display member may be at least partly wound around the rotating member. The display member may comprise a tape comprising indicia configured to display counting information.

The rotating member and the drive member may be configured to mechanically cooperate with one another. The drive member and the rotating member may be in direct mechanical contact, in particular engagement, with one another. The rotating member and the drive member may be configured to mechanically cooperate with one another such that rotation of the drive member in the first rotational direction is converted into the counting movement of the display member. Accordingly, movement, in particular axial movement, of the advancing member is converted into the rotational movement of the drive member and, thus, of the rotating member for driving the display member. In this way, provision of a very stable counter mechanism and, hence, a reliable drug delivery device is facilitated.

According to one embodiment, the drive member comprises a first driving feature, e.g. a toothing. The rotating member may comprise a second driving feature, e.g. a toothing. The driving features each may comprise a set of teeth. The driving features may be configured to engage with one another for transferring movement of the drive member in the first rotational direction into movement of the rotating member in the first rotational direction. Preferably, the rotating member and the drive member may be permanently engaged with one another. In this way, a very stable and reliable assembly may be provided.

According to one embodiment, the assembly further comprises a rotatable member. The rotatable member may comprise an unroll wheel. The rotatable member may comprise a pulled wheel. The display member may be adapted and arranged to mechanically cooperate with the rotatable member. The display member may be attached to, preferably permanently attached to, the rotatable member. The display member may be clamped between the rotatable member and the rotating member. The display member may be moveable between the rotatable member and the rotating member. In particular, the display member may perform the counting movement between the rotatable member and the rotating member. The drive member may be arranged between the rotatable and the rotating member. The drive member may be arranged between the rotatable and the rotating member such that the display member at least partly covers the drive member as seen in plan view onto the display member. However, the display member may be prevented from direct mechanical contact with the drive member. By means of the arrangement of the drive member between the rotatable and the rotating member, a compact and space-saving assembly may be provided.

According to an embodiment, a distance covered by the display member when the drive member is rotated in the first rotational direction, i.e. a distance the display member covers during its counting movement, may be determined by a distance between the teeth of the second interaction member. Alternatively, the distance covered by the display member when the drive member is rotated in the first rotational direction may be determined by a distance between the teeth of the respective driving feature and by a distance between the teeth of the second interaction member. Accordingly, the teeth of the second interaction member and/or the teeth of the respective driving feature may provide a mechanical advantage of the assembly.

The assembly may be adapted such that, when the drive member is rotated about a first angle, the display member may be rotated about a second angle. The second angle may be different from the first angle. In one embodiment, the second angle may be smaller than the first angle. For example, the first angle may amount to 90 degree and the second angle may be less than 90 degree, e.g. 60 degree, 50 degree, 45 degree, 30 degree or 15 degree. In an alternative embodiment, the second angle may be greater than the first angle. For example, the second angle may amount to 90 degree and the first angle may be less than 90 degree, e.g. 60 degree, 50 degree, 45 degree, 30 degree or 15 degree. In this way, a size of the indicia provided on the display member may be chosen dependent on the mechanical advantage provided by the assembly.

A further aspect relates to a drug delivery device. The drug delivery device may comprise the previously described assembly. The assembly may be integrated in the device. In this way, an improved, e.g. a compact and/or reliable and/or user-friendly device may be achieved.

Of course, features described above in connection with different aspects and embodiments may be combined with each other and with features described below.

In the following text, a set of advantageous aspects is described. The aspects are numbered to facilitate referencing features of one aspect in other aspects. Features from the aspects are not only relevant in connection with the specific aspects they relate to but are also of relevance on their own.

1. An assembly for a counter mechanism for a drug delivery device comprising
a drive member, wherein the drive member is adapted and arranged to be rotated in a first rotational direction and to be prevented from rotation in a second rotational direction which direction is opposite to the first rotational direction,
an advancing member adapted and arranged to be axially moved and rotated with respect to the drive member, wherein the advancing member is adapted and arranged to mechanically cooperate with the drive member such that the drive member is rotated in the first rotational direction,
a display member which is configured to count a number of doses and to display the counted number of doses,

wherein the assembly is adapted and arranged such that rotation of the drive member in the first rotational direction is converted into a counting movement of the display member.

2. The assembly according to aspect 1,

wherein the advancing member is configured to mechanically cooperate with the drive member when the advancing member is moved in a first axial direction with respect to the drive member, and wherein the advancing member is prevented from mechanical cooperation with the drive member when the advancing member is moved in a second axial direction with respect to the drive member, wherein the second axial direction is opposite to the first axial direction.

3. The assembly according to any of the previous aspects,

further comprising a plunger member which is moveable during at least one of a dose dispensing operation and a dose setting operation, wherein the plunger member is configured to mechanically cooperate with the advancing member such that the advancing member is axially moveable with respect to the drive member, wherein the plunger member comprises a first connection member and the advancing member comprises a second connection member, wherein the connection members are configured to mechanically cooperate with one another such that the plunger member is slideably connected to the advancing member.

4. The assembly according to aspect 3,

wherein the plunger member is axially moveable with respect to the advancing member between a first axial position and a second axial position, wherein,

(i) when the plunger member is arranged in the first axial position, movement of the plunger member in the first axial direction is converted into movement of the advancing member in the first axial direction due to mechanical cooperation of the connection members,

(ii) when the plunger member is arranged in the second axial position, movement of the plunger member in the second axial direction is converted into movement of the advancing member in the second axial direction due to mechanical cooperation of the connection members, and

(iii) when the plunger member is moved between the first axial position and the second axial position, the connection members are in a sliding engagement such that movement of the advancing member with respect to the drive member is prevented.

5. The assembly according to any of aspects 2 to 4,

wherein the advancing member comprises a first interaction member and the drive member comprises a second interaction member, wherein the interaction members are configured to mechanically cooperate with one another when the advancing member is moved in the first axial direction such that the first interaction member slides along the first interaction member, thereby exerting a force onto the drive member for rotating the drive member in the first rotational direction.

6. The assembly according to aspect 5,

wherein the interaction members are arranged offset with respect to one another before the interaction members mechanically cooperate when the advancing member is moved towards the drive member.

7. The assembly according to aspect 5 or aspect 6,

wherein the first interaction member comprises a set of teeth and wherein the second interaction member comprises a set of mating teeth, and wherein the interaction members are in a meshed engagement when the advancing member is positioned in an end position with respect to the drive member after the advancing member was moved towards the drive member.

8. The assembly according to any of aspects 2 to 7,

further comprising a centering feature, wherein the advancing member comprises an interaction feature adapted and arranged to prevent rotation of the advancing member with respect to the drive member when the advancing member is moved in the first axial direction due to mechanical cooperation with the centering feature, and wherein the interaction feature comprises a set of teeth.

9. The assembly according to any of aspects 2 to 8,

further comprising a reset feature, wherein the advancing member comprises a projection feature, wherein the reset feature and the projection feature are configured to mechanically cooperate with one another when the advancing member is moved in the second axial direction such that the reset feature and the projection feature slide along each other, the reset feature thereby exerting a force onto the advancing member for rotating the advancing member with respect to the drive member.

10. The assembly according to any of the previous aspects,

wherein the display member and the drive member are in direct mechanical contact with one another such that rotation of the drive member in the first rotational direction is converted into the counting movement of the display member.

11. The assembly according to any of aspects 1 to 9,

further comprising a rotating member which is coupled with the display member, wherein the rotating member and the drive member are configured to mechanically cooperate with one another such that rotation of the drive member in the first rotational direction is converted into the counting movement of the display member.

12. The assembly according to aspect 11,

wherein the drive member comprises a first driving feature and the rotating member comprises a second driving feature and wherein the driving features are configured to engage with one another for transferring movement of the drive member in the first rotational direction into movement of the rotating member in the first rotational direction.

13. The assembly according to any of the previous aspects,

wherein the assembly is adapted such that, when the drive member is rotated about a first angle, the display member is rotated about a second angle, wherein the second angle is different from the first angle.

14. The assembly according to any of the previous aspects,

wherein the assembly is adapted such that in plan view onto the display member (13), the display member covers the drive member at least partly.

15. A drug delivery device comprising the assembly according to one of aspects 1 to 14.

### BRIEF DESCRIPTION OF THE FIGURES

Further features and refinements become apparent from the following description of the exemplary embodiments in connection with the accompanying figures.

FIGS. 1A and 1B schematically show a perspective view of a drug delivery device,

FIG. 1C schematically shows a sectional side view of the drug delivery device of FIGS. 1A and 1B,

FIGS. 2A, 2B, 2C, 2D, 2E, 2F and 2G schematically show a perspective view of parts of the drug delivery device of FIGS. 1A to 1C,

FIGS. 3A and 3B schematically show a sectional side view of parts of the drug delivery device of FIGS. 1A to 1C,

FIGS. 4A and 4B schematically show a perspective view of a part of the drug delivery device of FIGS. 1A to 1C,

FIG. 5 schematically shows a perspective view of a part of the drug delivery device of FIGS. 1A to 1C according to a first embodiment,

FIG. 6 schematically shows a perspective view of a part of the drug delivery device of FIG. 1A to 1C according to the first embodiment,

FIG. 7 schematically shows a sectional side view of a part of the drug delivery device of FIGS. 1A to 1C according to the first embodiment,

FIG. 8 schematically shows a sectional side view of a part of the drug delivery device of FIGS. 1A to 1C according to the first embodiment,

FIG. 9 schematically shows an exploded view of parts of the drug delivery device of FIGS. 1A to 1C according to a second embodiment,

FIG. 10 schematically shows a perspective view of parts of the drug delivery device of FIGS. 1A to 1C according to the second embodiment,

FIGS. 11A and 11B schematically show a perspective view of parts of the drug delivery device of FIGS. 1A to 1C according to the second embodiment,

FIGS. 12A and 12B schematically show a perspective view of parts of the drug delivery device of FIGS. 1A to 1C according to the second embodiment,

FIGS. 13A and 13B schematically show a perspective view of parts of the drug delivery device of FIGS. 1A to 1C according to the second embodiment,

FIGS. 14A, 14B, 14C, 14D, 14E, 14F, 14G and 14I schematically show a perspective view or a sectional side view of parts of the drug delivery device of FIGS. 1A to 1C according to the second embodiment.

Like elements, elements of the same kind and identically acting elements may be provided with the same reference numerals in the figures.

### DETAILED DESCRIPTION

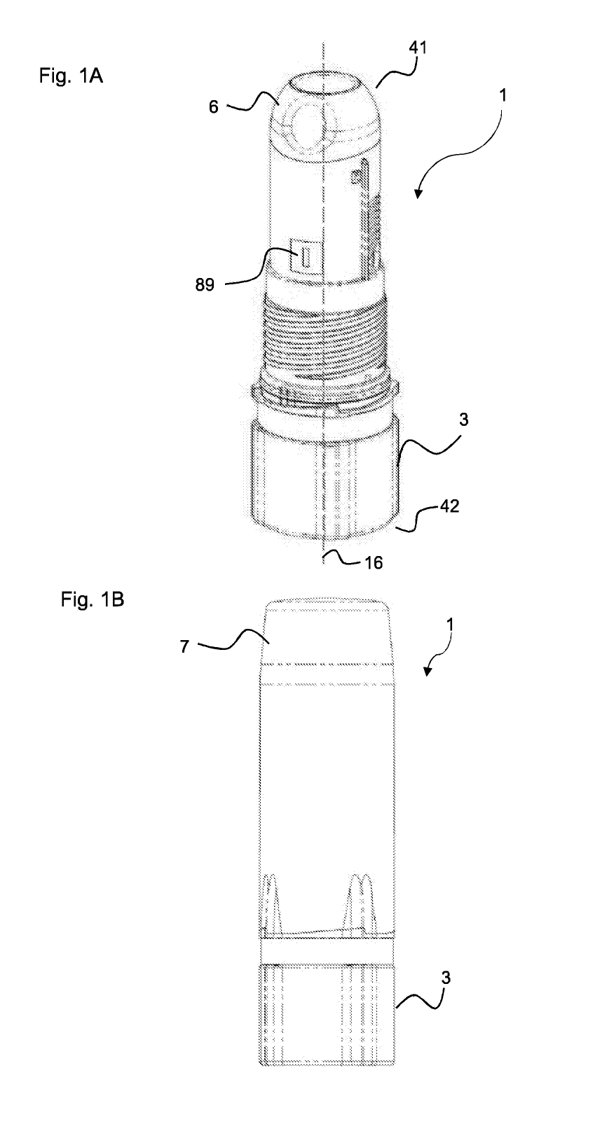

In FIGS. 1A, 1B and 1C, an inhalation device 1 is shown. The inhalation device 1 comprises a housing 3. The device 1 comprises an outer cylinder 4. The outer cylinder 4 is secured against axial movement with respect to the housing 3. The outer cylinder 4 is rotatable with respect to the housing 3. The inhalation device 1 further comprises a mouthpiece 6. The device 1 and the housing 3 have a distal end 41 and a proximal end 42. The term “distal end” designates that end of the device 1 or a component thereof which is or is to be arranged closest to the mouthpiece 6. The term “proximal end” designates that end of the device 1 or a component thereof which is or is to be arranged furthest away from the mouthpiece 6. The distal end 41 and the proximal end 42 are spaced apart from one another in the direction of an axis 16. The axis 16 may be the main longitudinal axis or rotational axis of the device 1.

The inhalation device 1 comprises a cap 7. The cap 7 is used for covering the mouthpiece 6. In FIGS. 1B and 1C, the cap 7 is mounted onto the device 1 for covering the mouthpiece 6. In FIG. 1A, the cap 7 is removed from the device 1. The cap 7 may comprise a thread, preferably a screw thread. The cap 7 may be rotatable with respect to the housing 3 for screwing the cap 7 onto the device 1 and for unscrewing the cap 7 from the device 1. The outer cylinder 4 is rotationally fixed to the cap 7. In particular, the outer cylinder 4 follows rotation of the cap 7 with respect to the housing 3. For the detailed description of the components of the inhalation device 1 and their mechanical cooperation it is referred to document WO 2009/065707 A1.

The device 1 comprises a storage chamber 15. The storage chamber 15 holds one dose, preferably a plurality of doses, of a medical substance 2. The substance 2 may be a powder. In particular, the plurality of doses may correspond to a predefined number of doses, such that after the predefined number of doses has been delivered a lock-out mechanism (not explicitly shown in the Figures) may prevent a further operation of the device.

A numerical value corresponding to the predefined number of doses is a starting value of a counter mechanism. Before delivery of a first dose, the counter mechanism displays this predefined number as the number of available doses and with every dose delivery the number is decremented. Alternatively, the counter mechanism may display the number of doses that already have been delivered. In this case, the counter mechanism displays “0” as predefined number before delivery of the first dose and with every dose delivery the number is incremented. The counter mechanism is not shown in FIGS. 1A, 1B, 1C, but the dose counter mechanism will be discussed later on in detail.

The storage chamber 15 is terminated by a chamber ceiling 24. The chamber sealing 24 is formed integrally with a top wall of the storage chamber 15. The device 1 further comprises a rotary part 25. The rotary part 25 is of substantially plate-like configuration and is connected in a rotationally fixed manner to the outer cylinder 4. Accordingly, the rotary part 25 follows rotation of the cap 7 and, hence, of the outer cylinder 4 about the main longitudinal axis or rotational axis 16 of the device 1 with respect to the storage chamber 15. However, the rotary part 25 is axially fixed relative to the housing 3.

The device 1 further comprises a metering rod 33. The metering rod 33 may be connected to the cap 7 by a snap fit element 34 when the cap 7 is engaged to the housing 3. When the cap 7 is re-engaged to the housing 3, the metering rod 33 travels axially in the proximal direction such that a most proximal part of the metering rod 33 comprising a metering chamber 40 enters the storage chamber 15. When the cap 7 is disengaged from the housing 3 the metering rod 33 travels axially in the distal direction such that the most proximal part of the metering rod 33 exits the storage chamber 15. The metering rod 33 is configured for functioning as a moving metering chamber 40 for a sub-quantity 14 of the substance 2 which is to be dispensed during a specific delivery action. The metering chamber 40 is provided in that end section of the metering rod 33 which projects into the substance 2, e.g. the proximal end section.

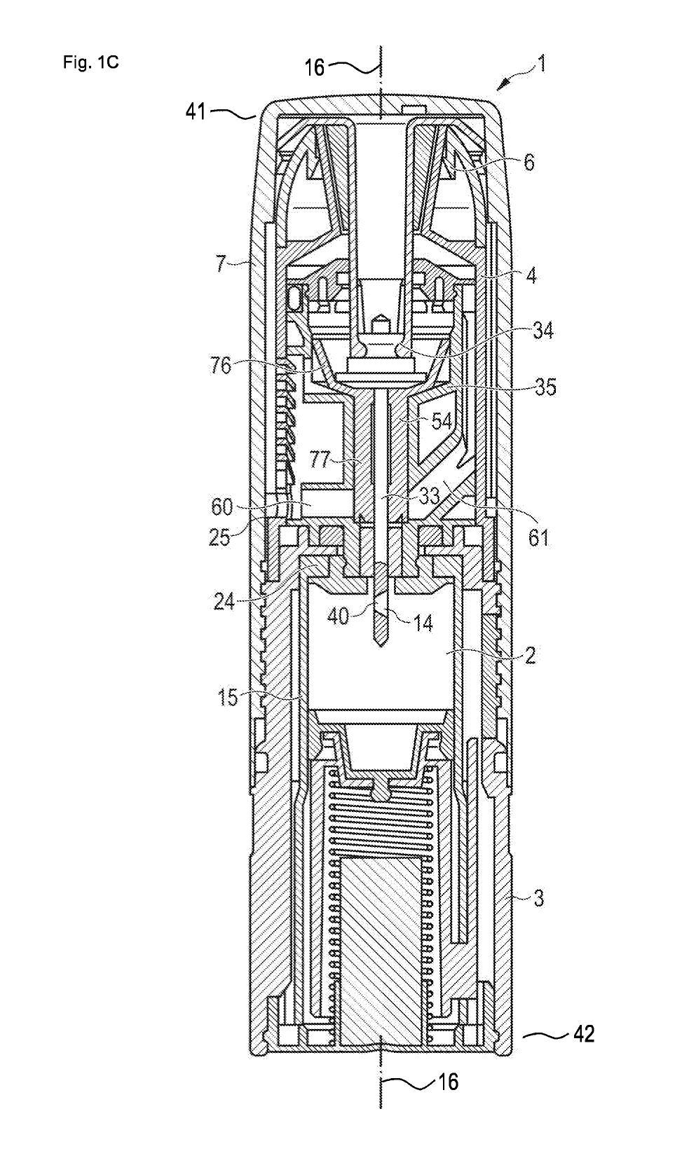

The inhalation device 1 further comprises a flow path comprising a flow channel 60 and an intermediate channel portion 61. The inhalation device 1 further comprises an actuating element 54 (see also FIG. 2A). The actuating element 54 is arranged within an actuator housing 35 (see also FIG. 2B). The actuator housing 35 is configured substantially as a hollow body. The actuator housing 35 is secured against axial movement with respect to the housing 3. The actuator housing 35 is adapted and arranged to guide an axial movement of the actuating element 54. The actuator housing 35 is arranged within the inner cylinder 4. The actuating element 54 and the actuator housing 35 are prevented from rotation with respect to one another.

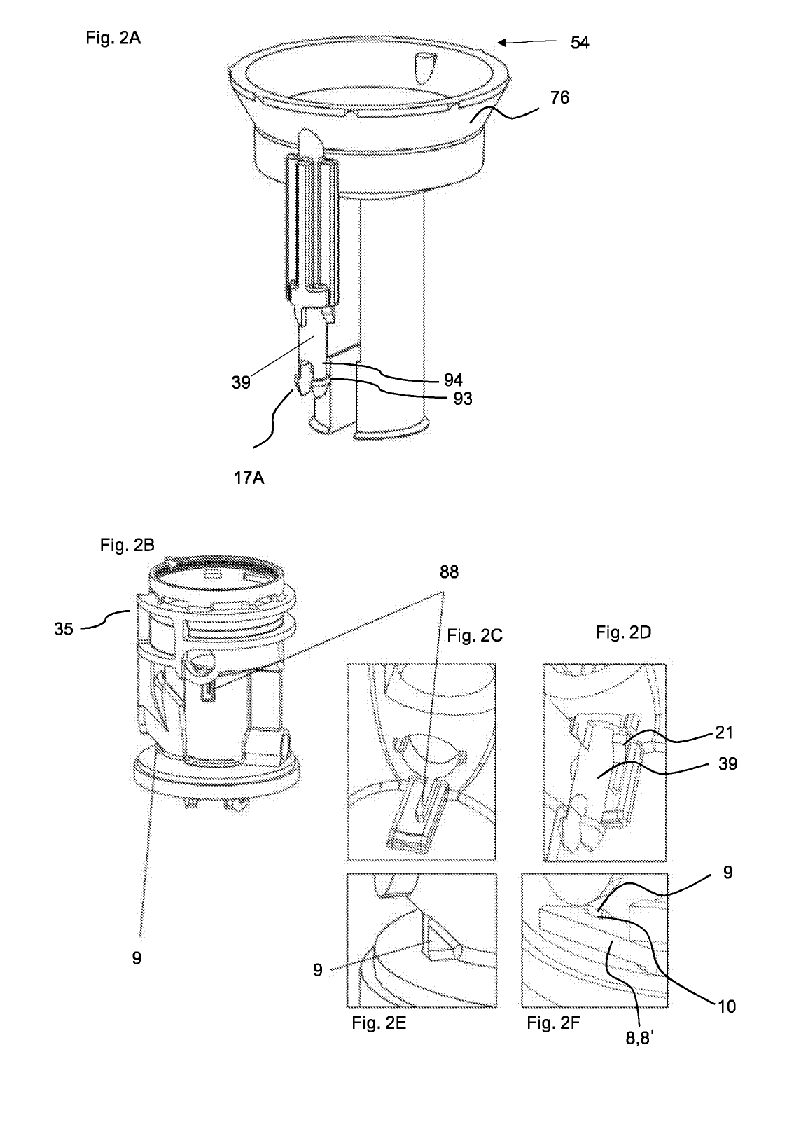

The actuating element 54 comprises a piston comprising tongues 77 and a head 76. The actuating element 54 further comprises a plunger member 39, in particular a plunger (see, for example, FIGS. 2A, 2D, 3A and 3B). The plunger 39 is guided through an opening 88 in the actuator housing 35 (see FIGS. 2C and 2D). A first end of the plunger member 39 is attached to the head 76 (see FIG. 2A). A second end of the plunger member 39 is free from mechanical cooperation with the actuating element 54. The plunger member 39, in particular the second end, comprises a first connection member 17A (see FIG. 2A). The first connection member 17A may comprise at least one protrusion, for example. The function of the first connection member 17A is explained later on in detail. The head 76 of the actuating element 54 is formed from a soft material which can be deformed easily. The plunger member 39 is formed from a material which is harder than the soft material of the head 76. Thus, the plunger member 39 may be moveable relative to the head 76 of the actuating element 54 in various directions. The plunger member 39 and the head 76 are formed in a two-component molding process. The soft material of the head 76 is molded around the first end of the plunger member 39.

The actuating element 54 has a first and a second position. The first position is more proximal than the second position. In the first position, the tongues 77 of the actuating element 54 are configured to block the flow path between the flow channel 60 and the intermediate channel portion 61. In the second position, the actuating element 54 is positioned more distally, i.e. closer to the mouthpiece 6, such that the tongues 77 no longer block the flow path between the flow channel 60 and the intermediate channel portion 61.

The cap 7 is removed from the housing 3 by unscrewing the cap 7 from the housing 3. Accordingly, the cap 7 performs a concurrent axial movement in the distal direction and a rotational movement. The cap 7 and the rotary part 25 are in a splined engagement when the cap 7 is attached to the housing 3. During disengagement of the cap 7 from the housing 3, the rotational movement of the cap 7 is transferred into a rotation of the rotary part 25 around the longitudinal axis 16 due to their splined engagement. The rotation of the rotary part 25 is transferred into a rotation of the actuating element 54. Furthermore, the concurrent axial and rotational movement of the cap 7 is transferred to the metering rod 33 concurrently performing an axial movement in the distal direction and a rotational movement around the longitudinal axis 16. As the cap 7 approaches the end of the threaded connection to the housing 3, the snap fit element 34 disengages from the metering rod 33. During disengagement of the cap 7 from the housing 3, the actuating element 54 is not moved in an axial direction with respect to the housing 3. Accordingly, the actuating element 54 is in the first position before and after disengagement of the cap 7 from the housing 3.

When the cap 7 is fully disengaged from the housing 3, the metering chamber 40 is in a first condition. The first condition of the metering chamber 40 is defined by the tongues 77 of the actuating element 54 closing the metering chamber 40 such that the metering chamber 40 is not in contact with the flow path. Accordingly, when the actuating element 54 is in the first position and the cap 7 is disengaged from the housing 3, the metering chamber 40 is in the first condition. In the first condition of the metering chamber 40, the tongues 77 of the actuating element 54 cover the metering chamber 40 on each side. Accordingly, in this first condition, it is not possible for the sub-quantity 14 of substance to trickle out. Rather, the substance 2 is reliably retained in the metering chamber 40.

After the cap 7 has been demounted, the user may trigger an inhalation operation by subjecting the device to a suction airstream, in the simplest case by the user breathing in. Air is sucked in via the mouthpiece 6, and this, in first instance, by virtue of the head 76 being subjected to the action of air, results in the actuating element 54 being displaced axially towards the mouthpiece 6, i.e. in the distal direction. By virtue of the axially displaced actuating element 54, the tongues 77 are likewise displaced axially, in order to release the metering chamber 40. The metering chamber 40 is then in a second condition. The second condition of the metering chamber 40 is defined by the actuating element 54 being in the second position. In the second condition, the metering chamber 40 lies freely in the flow path between the flow channel 60 and the intermediate channel portion 61. The metering chamber 40 is cleared out with air being sucked from the flow channel 60.

After the inhalation operation was finished, the cap 7 can be re-engaged to the housing 3. During engagement of the cap 7 to the housing 3, the cap 7 is moved axially in the proximal direction and concurrently rotated around the longitudinal axis 16. The snap fit element 34 engages to the metering rod 33 at the beginning of the threaded connection. Thereby, the metering rod 33 is rotated and moved into the proximal direction when the cap 7 is engaged to the housing 3. During re-engagement of the cap 7 to the housing 3, the metering rod 33 is moved in the proximal direction due to the interaction with the cap 7. Movement of the metering rod 33 in the proximal direction is transferred to the actuating element 54 (if the actuating element 54 is in the second position). Thereby, the actuating element 54 is moved from the second position to the first position. However, in case that the cap 7 is disengaged from the housing 3 and, afterwards, re-engaged to the housing 3 without a drug delivery being performed in the meantime, the actuating element 54 remains in the first position the whole time. Accordingly, as the actuating element 54 is already in the first position, it cannot be moved axially during engagement of the cap 7 to the housing 3.

The inhalation device 1 further comprises a dose counting or counter mechanism 5 (see FIGS. 2G, 3A, 3B, 5, 6, 7, 8, 9, 10 and 14A to 14I, for example). The mechanism 5 is arranged within the device 1 and, in particular, within the inner cylinder 4 of the device 1 (see FIGS. 3A and 3B). The mechanism 5 may be connected to the actuator housing 35 as described later on in more detail and as shown in FIG. 2G. The mechanism 5 is configured to count the number of doses left in the device 1. The mechanism 5 is further configured to display the counted number corresponding e.g. to the number of doses left in the device 1. Alternatively, the counted number may correspond to another quantity, e.g. the number of doses which have been delivered by the inhalation device 1.

The mechanism 5 comprises a body 8, 8′ (see, for example, FIGS. 2G, 5, 6, 7, 8, 9, 11A, 11B and 14A). The body 8, 8′ is configured to be snap-fitted to the actuator housing 35 of the inhalation device 1. For this purpose, the body 8, 8′ and the actuator housing 35 comprise corresponding mounting elements 9, 10, e.g. snap-fit elements as can be gathered from FIGS. 2B, 2E and 2F, for example. The mounting elements 9, 10 may comprise at least one protrusion and at least one corresponding indentation. Due to mechanical cooperation of the mounting elements 9, 10, the body 8, 8′ is fixed to the actuator housing 35.

The mechanism 5 further comprises a display member 13, which is shown in FIGS. 2G, 5, 7, 8, 10, 14A, 14G and 14I, for example. The display member 13 is a counting member configured to count and to display the counted number. The display member 13 comprises a tape a foil. A thickness of the display member may amount to 12 μm, for example. The display member 13 comprises numbers or indicia arranged on the display member 13. In particular, the numbers are arranged on the display member 13 such that one number is visible in a window 89 (see FIG. 1A) of the rotary body 25 in each position of the display member 13. This number corresponds to the counted number of the mechanism 5.

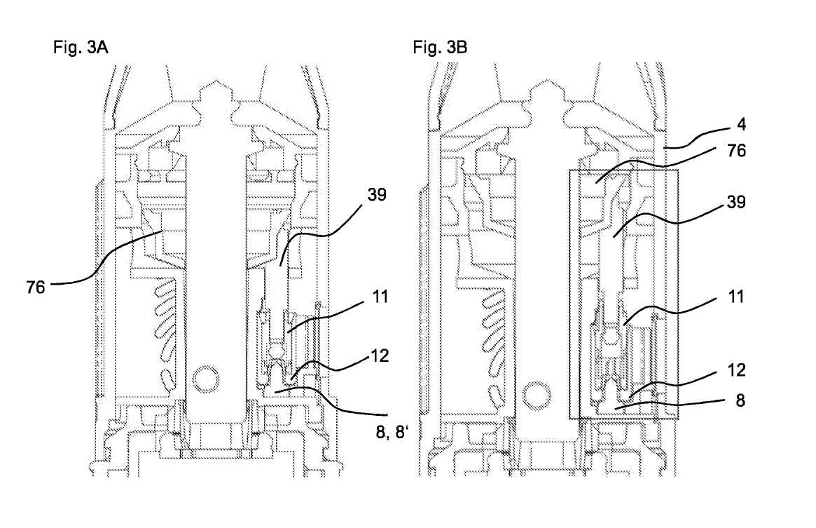

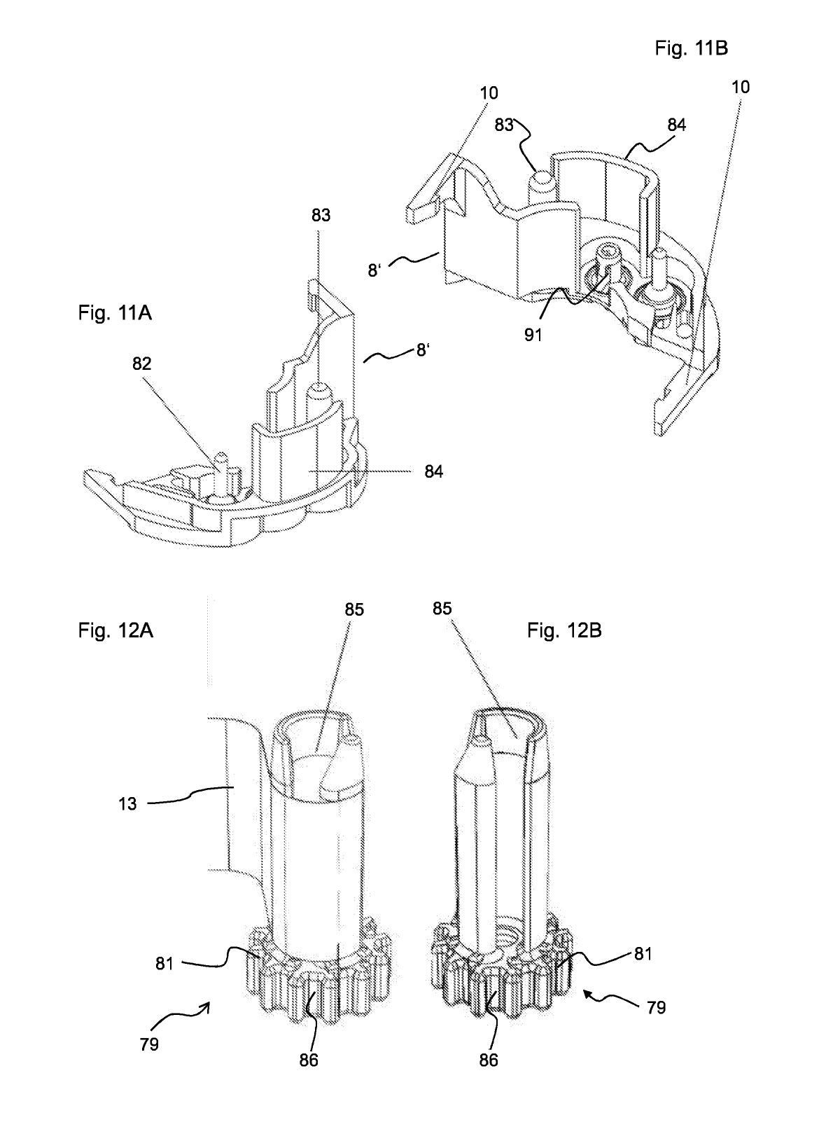

The mechanism 5 further comprises a rotatable member 32, which is shown in FIGS. 5, 6, 14B, 14G and 14H, for example. The body 8, 8′ comprises a mounting element 83 (see, for example, FIGS. 11A and 11B). The mounting element 83 may comprise a pin. The mounting element 83 is configured for mounting the rotatable member 32 to the body 8, 8′.

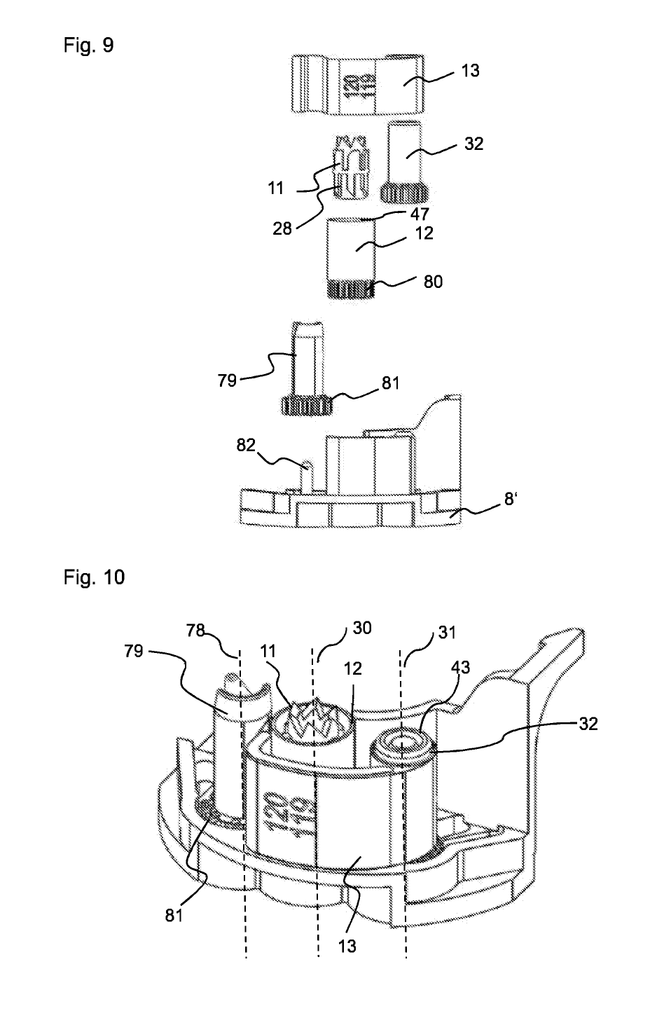

The rotatable member 32 comprises a pulled wheel or unroll wheel. The rotatable member 32 is configured to rotate around a second rotational axis 31 (see FIGS. 5 and 10). The second rotational axis 31 is parallel to the main longitudinal axis 16. One end of the display member 13 is attached to the rotatable member 32, in particular to a body or body part of the rotatable member 32. The one end may be fixed to the rotatable member 32 or may be releasably attached to the rotatable member 32. In this context, the term “one end being fixed to the rotatable member 32” means that the one end can only be removed from the rotatable member 32 by damaging the display member 13.

The mechanism 5 comprises a first state and a second state. In the first state of the mechanism 5, the display member 13 is wound around the rotatable member 32 such that the display member 13 covers a first surface 43 of the rotatable member 32 (see, for example, FIGS. 5 and 10). A surface normal of the first surface 43 is perpendicular to the second rotational axis 31. The display member 13 is configured to cover the first surface 43 at least partially. In the first state of the mechanism 5, the display member 13 covers the first surface 43 at least partly. In the second state of the mechanism 5, the display member 13 covers a smaller part of the first surface 43 than in the first state of the mechanism 5.

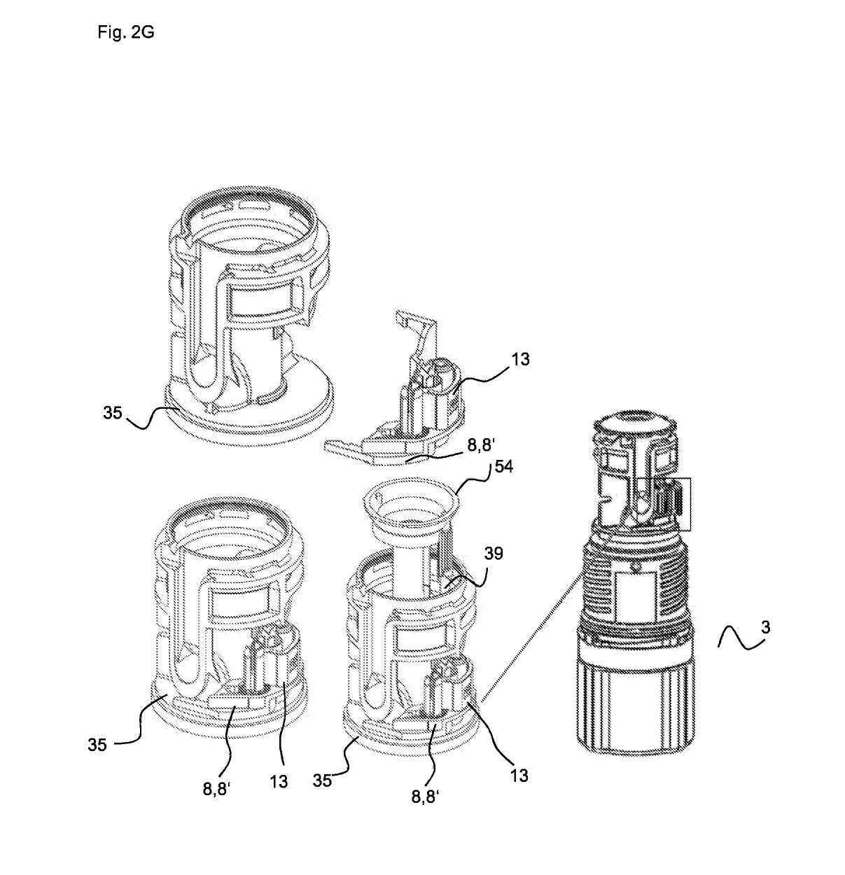

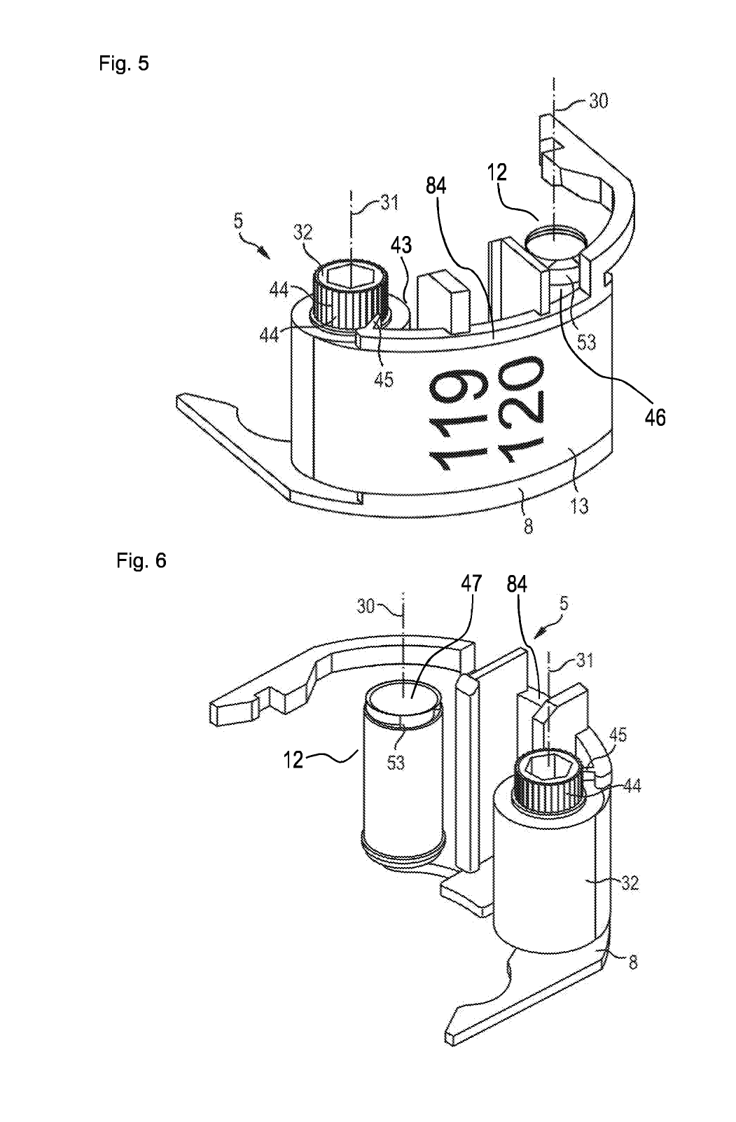

Furthermore, the mechanism 5 comprises a drive member 12, which is shown in FIGS. 3A, 3B, 4A, 4B, 5, 6, 7, 8, 9, 10, 14A, 14B, 14C, 14D, 14E, 14F, 14G, 14H and 14I. The drive member 12 is also a rotatable member. The drive member 12 may comprise a drum. The body 8, 8′ comprises a mounting element 91 (see, for example, FIGS. 7 and 11B). The mounting element 91 may comprise a pin or protrusion. The mounting element 91 is configured for mounting the drive member 12 to the body 8, 8′. For this purpose, the drive member 12 comprises a mating mounting element 92, e.g. an indentation (see FIGS. 4B and 7, for example).

According to a first embodiment, which is shown in the FIGS. 5, 6, 7 and 8, the display member 13 mechanically cooperates, in particular directly mechanically cooperates, with the drive member 12. In particular, in this embodiment, the second end of the display member 13 is attached to the drive member 12. The second end is attached either by a fixed attachment or by a releasable attachment to the drive member 12, which is described later on in detail.

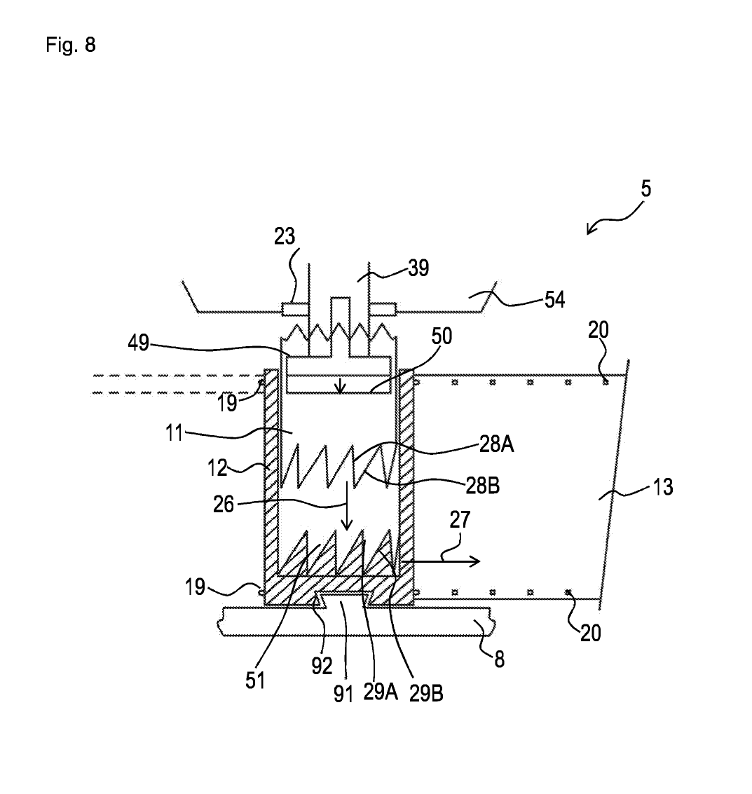

According to a second embodiment, which is shown in the FIGS. 9, 10, 11A, 11B, 12A, 12B, 13A, 13B and 14A to 14I, the display member 13 may be prevented from direct mechanical contact with the drive member 12. In particular, the display member 13 may be passed along the drive member 12 without having direct mechanical contact with the drive member 12, which is explained later in more detail.

The drive member 12 is adapted and arranged to rotate around a third rotational axis 30 (see FIGS. 5 and 10). The third rotational axis 30 is parallel to the second rotational axis 31. The third rotational axis 30 is parallel to the main longitudinal axis 16. In both embodiments, the display member 13 is mounted to the rotatable member 32 such that, in response to a rotation of the drive member 12 in a first rotational direction, the display member 13 performs a counting movement. Due to the counting movement, a succeeding number of doses which can still be dispensed or which have already been dispensed is displayed by the display member 13. Thereby, in response to a rotation of the drive member 12 in the first rotational direction, the display member 13 is unwound from the rotatable member 32. As the display member 13 is unwound from the rotatable member 32, the rotatable member 32 is rotated in the first rotational direction. The body 8, 8′ may comprise a guidance face or support member 84 (see FIGS. 5, 6, 11A and 11B) configured to guide the display member 13 when the display member 13 is at least partly unwound from the rotatable member 32.

According to the first embodiment, upon rotation of the drive member 12 in the first rotational direction, the display member 13 is wound around the drive member 12.

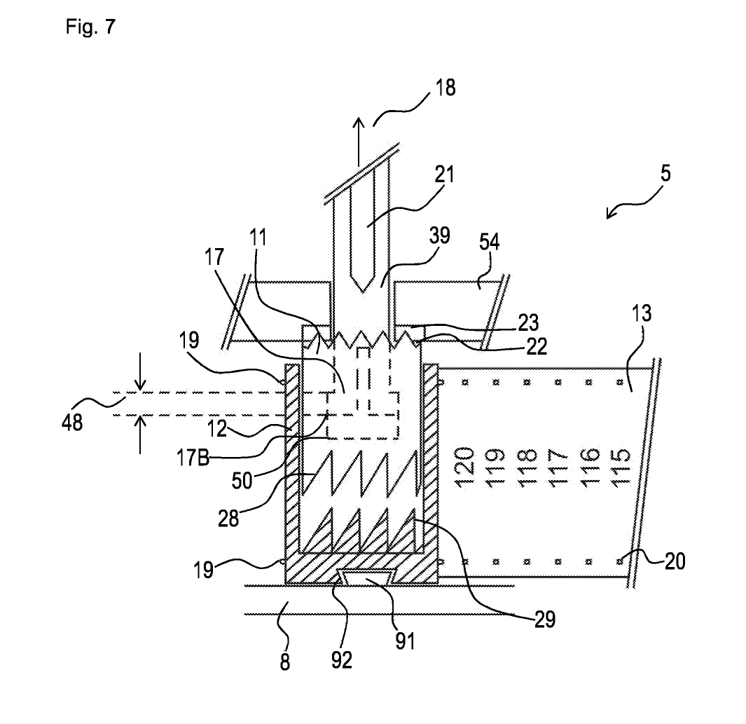

The drive member 12 comprises a first surface 46 constructed in the same way as the first surface 43 of the rotatable member 32 (see FIG. 5). The first surface 46 is configured such that the display member 13 may be wound up on the first surface 46. For winding the display member 13 around the drive member 12 when the drive member 12 is rotated in the first rotational direction, the drive member 12 comprises a plurality of drive features 19 (see FIGS. 7 and 8). The drive features 19 are arranged on an outer surface of the drive member 12. The drive features 19 are arranged around the drive member 12. The drive features 19 are arranged in pairs. The respective pair of drive features 19 is arranged along an axis which is parallel to the third rotational axis 30. One drive feature 19 of a pair is arranged in a proximal end section of the drive member 12. The further drive feature 19 of that pair is arranged in a distal end section of the drive member 12. Succeeding pairs of the drive features 19 may be equidistant with respect to one another. The respective drive feature 19 comprises a protrusion. The drive feature 19 protrudes from the drive member 12 in a radial outward direction. The drive feature 19 protrudes in a direction which is perpendicular to the third rotational axis 30.

The display member 13 comprises a plurality of interactions features 20. The interaction members 20 may be arranged in pairs. One interaction member 20 of a pair is arranged in the distal end section of the display member 13. The further interaction member 20 of the pair is arranged in the proximal end section of the display member 13. The respective pair of interaction members 20 is arranged along an axis which is parallel to the third rotational axis 30. The display member 30 comprises a plurality of pairs of interaction members 20. Succeeding pairs of the interaction members 20 may be equidistant with respect to one another. The respective interaction member 20 comprises a perforation or protrusion of the display member 13. The pairs of interaction members 20 are adapted and arranged to mechanically cooperate with the drive features 19, in particular with the pairs of drive features 19 of the drive member 12. In particular, one pair of interaction members 20 is configured to engage one pair of the drive features 19 at a time. When the drive member 12 is rotated in the first rotational direction, a succeeding pair of drive features 19 may engage a succeeding pair of interaction members 20 to wind the display member 13 further around the drive member 12.

When the display member 13 is unwound from the rotatable member 32, a diameter of the rotatable member 32 decreases. When the display member 13 is wound on the drive member 12, a diameter of the drive member 12 increases. In this context, the diameter of the rotatable member 32 is measured in a direction perpendicular to the second rotational axis 31. Furthermore, the term “diameter of the rotatable member 32” shall refer to the diameter of the rotatable member 32 including the part of the display member 13 wound around the rotatable member 32. Respectively, the diameter of the drive member 12 is measured in a direction perpendicular to the third rotational axis 30. The term “diameter of the drive member 12” shall refer to the diameter of the drive member 12 including the part of the display member 13 wound around the drive member 12.

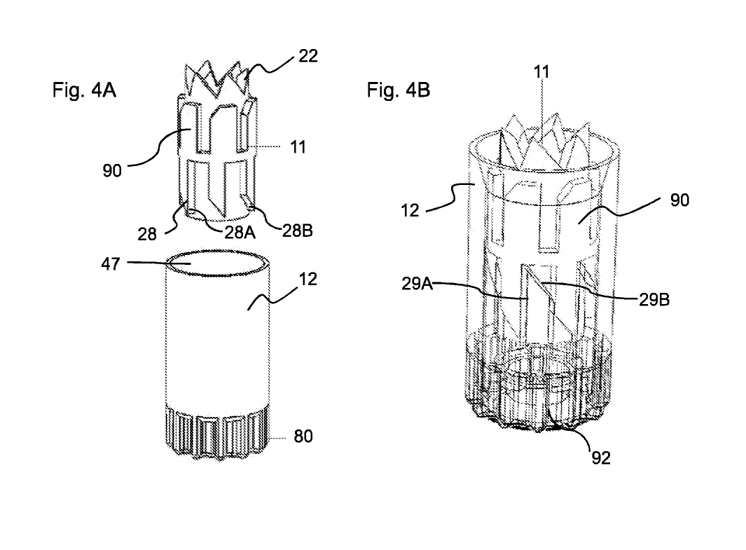

In this first embodiment, the rotatable member 32 may comprise a reverse rotation prevention feature 44 (see FIGS. 5 and 6) which is configured to permit a rotation of the rotatable member 32 in the first rotational direction and which is further configured to prevent a rotation of the rotatable member 32 in a second rotational direction which is opposite to the first rotational direction. The reverse rotation prevention feature 44 may comprise teeth arranged on the rotatable member 32. The teeth are configured to cooperate with a pawl 45 of the body 8 (see FIGS. 5 and 6). The reverse rotation prevention feature 44 may comprise a snap action mechanism. The reverse rotation prevention feature 44 and the pawl 45 are shaped such that a rotation of the rotatable member 32 in the second rotational direction is prevented. Furthermore, the reverse rotation prevention feature 44 and the pawl 45 are shaped such that a rotation of the rotatable member 32 in the first rotational direction is allowed. Alternatively, rotation of the rotatable member 32 in the first rotational direction as well as in the second rotational direction may be enabled in this embodiment. In this case, the reverse rotation prevention feature 44 and the pawl 45 may be redundant.

Moreover, the drive member 12 comprises a reverse rotation prevention feature 53. The reverse rotation prevention feature 53 of the drive member 12 is configured to permit a rotation of the drive member 12 in the first rotational direction and is further configured to prevent a rotation of the drive member 12 in the second rotational direction. The reverse rotation prevention feature 53 may be constructed in the same way as the reverse rotation prevention feature 44 of the rotatable member 32. In particular, the reverse rotation prevention feature 53 of the drive member 12 may comprise teeth arranged on the drive member 12 which are configured to cooperate with a further pawl (not explicitly shown in the Figures) of the body 8. The reverse rotation prevention feature 53 of the drive member 12 and the further pawl are shaped such that a rotation of the drive member 12 in the second rotational direction is prevented. Furthermore, the reverse rotation prevention feature of the drive member 12 and the further pawl are shaped such that a rotation of the drive member 12 in the first rotational direction is allowed.

According to the second embodiment, the mechanism 5 further comprises a rotating member 79, which is, for example, shown in FIGS. 9 and 10. The body 8′ comprises a mounting element 82 (see, for example, FIGS. 11A and 11B). The mounting element 82 may comprise a pin. The mounting element 82 is configured for mounting the rotating member 79 to the body 8′.

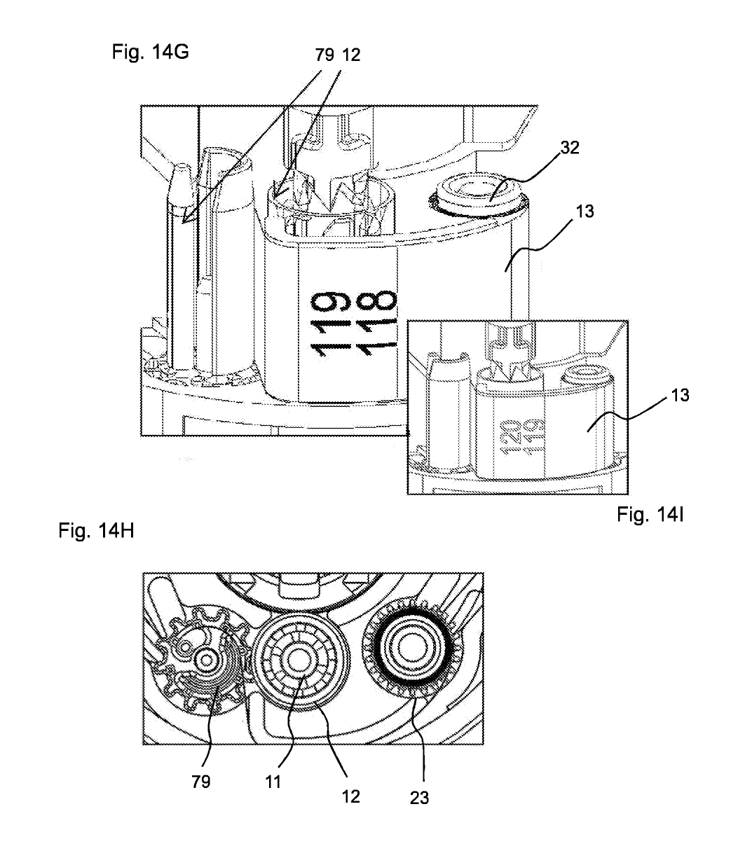

In this second embodiment, the drive member 12 is arranged between the rotatable member 32 and the rotating member 79 (see, for example, FIGS. 10, 14A, 14B, 14G, 14H, 14I). The drive member 12 is arranged between the rotatable member 32 and the rotating member 79 such that, as seen in plan view onto the display member 13, the display member 13 at least partly covers the drive member 12. However, the display member 13 may be prevented from direct mechanical contact with the drive member 12 as described above. In particular, direct mechanical contact between the drive member 12 and the display member 13 may be prevented by means of the support member 84.

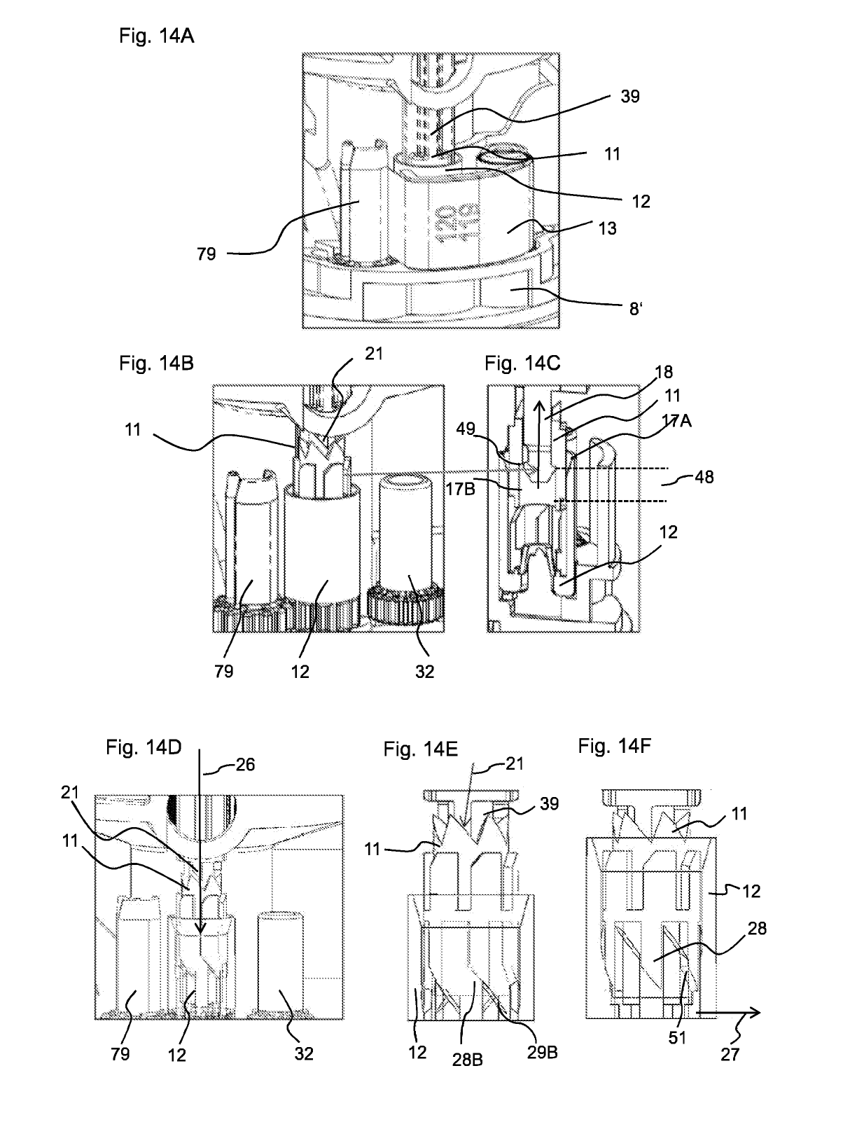

The rotating member 79 is rotatable in the first rotational direction. The rotating member 79 is rotatable about a forth rotational axis 78, in particular a longitudinal axis of device 1 (see FIG. 10). The forth rotational axis 78 is parallel to the main longitudinal axis 16. The rotating member 79 is prevented from rotation in the second rotational direction. For this purpose, the mechanism 5 comprises a pawl member 87 (see FIGS. 13A and 13B). The rotating member 79 is configured to cooperate with the pawl member 87 such that a rotation of the rotating member 79 in the first rotational direction is allowed and such that rotation of the rotating member 79 in the second rotational direction is prevented. For this purpose, the rotating member 79 comprises a toothing 81 which mechanically cooperates with the pawl member 87 and which is referred to later on in more detail.

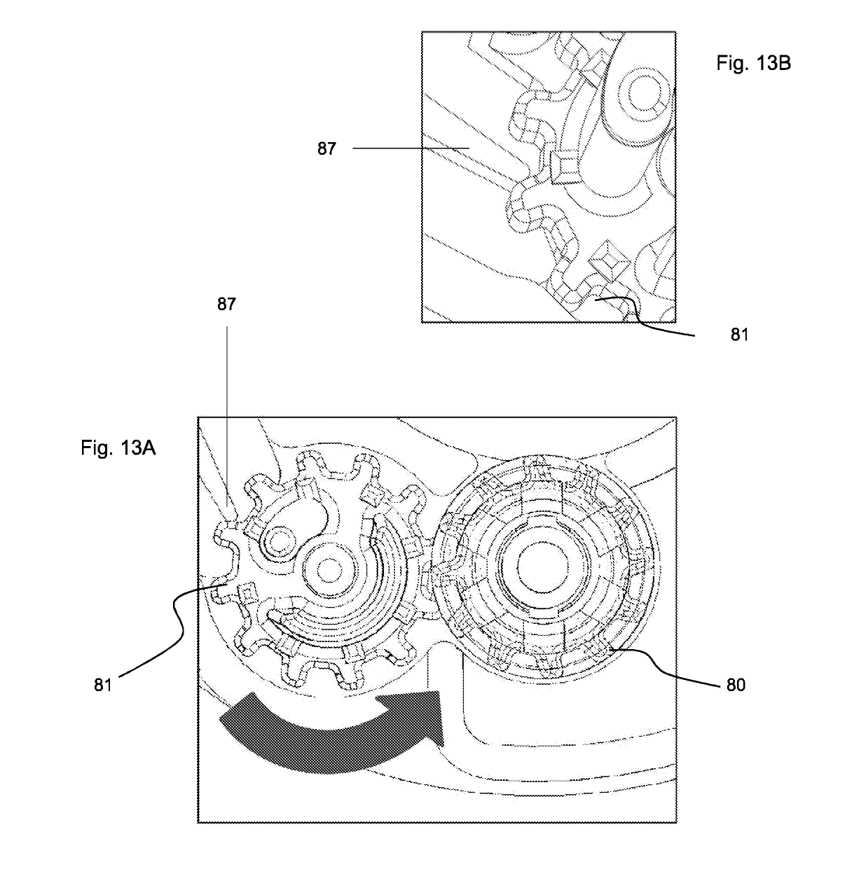

The rotating member 79 is prevented from axial movement. The rotating member 79 is coupled with the display member 13. In particular, the display member 13 is attached to, preferably non-releasably attached to, the rotating member 79, as shown in FIGS. 10 and 12A, for example. The display member 13 is at least partly wound around the rotating member 79. The rotating member 79 may comprises a wheel member or wheel 86 (see FIGS. 12A and 12B). The rotating member 79 may further comprise a body member 85. The wheel 86 and the body member 85 may be non-releasably connected to one another. The display member 13 is preferably attached to the body member 85. The body member 85 is adapted and arranged for rolling up the display member 13 upon rotation of the drive member 12 in the first rotational direction as described below in detail (see FIG. 12A).

The rotating member 79 and the drive member 12 are configured to mechanically cooperate with one another. The drive member 12 and the rotating member 79 are in direct mechanical contact, in particular engagement with one another. The rotating member 79 and the drive member 12 mechanically cooperate with one another such that rotation of the drive member 12 in the first rotational direction is converted into the counting movement of the display member 13. For this purpose, the drive member 12 comprises a first driving feature 80 (see, for example, FIG. 9), e.g. a toothing. The first driving feature 80 is arranged on an outer surface of the drive member 12. The first driving feature 80 is arranged in a proximal end section of the drive member 12. The rotating member 79 and, in particular the wheel 86, comprises a second driving feature 81 (see FIGS. 9, 12A and 12B, for example). The second driving feature 81 may correspond to the previously mentioned toothing. The second driving feature 81 is arranged on an outer surface of the rotating member 79. The second driving feature 81 is arranged in a proximal end section of the rotating member 79. The driving features 80, 81 are configured to engage with one another for transferring the rotational movement of the drive member 12 in the first rotational direction into movement of the rotating member 79 in the first rotational direction.

Upon rotation of the drive member 12 in the first rotational direction, the display member 13 is unwound from the rotatable member 32. Thereby, the display member 13 is lead along the support member 84, thereby passing the drive member 12 (see, for example, FIGS. 10, 14A and 14G). Upon rotation of the drive member 12 in the first rotational direction, the display member 13 is wound around the rotating member 79. The rotating member 79 comprises a first surface (not explicitly shown in the Figures) constructed in the same way as the first surface 43 of the rotatable member 32 (see FIG. 5 in this context). The first surface of the rotating member 79 is configured such that the display member 13 may be wound up on the first surface.

When the display member 13 is unwound from the rotatable member 32, a diameter of the rotatable member 32 decreases. When the display member 13 is wound on the rotating member 79, a diameter of the rotating member 79 increases. In this context, the diameter of the rotating member 79 is measured in a direction perpendicular to the longitudinal axis around which the rotating member 79 is rotated, i.e. the forth rotational axis 78. Furthermore, the term “diameter of the rotating member 79” shall refer to the diameter of the rotating member 79 including the part of the display member 13 wound around the rotating member 79.

In the second embodiment, the rotatable member 32 may not comprise a reverse rotation prevention feature. In particular, the rotatable member 32 may be rotatable in the first and in the second rotational direction. By rotating the rotatable member 32 in the first and in the second rotational direction, the display member 13 can be stretched, for example.

In the following, the operation of the mechanism 5 is described in detail. The operation of the mechanism 5 is the same for the first and for the second embodiment. The mechanism 5 and, in particular the drive member 12, is configured to cooperate with a dosing mechanism of the device 1 which allows for a dose setting and/or a dose dispensing operation of the inhalation device 1. The dosing mechanism comprises the previously described actuating element 54, which is shown in FIG. 2A.

The drive member 12 may be a hollow member. In particular, the drive member 12 comprises an opening 47 (see FIGS. 4A, 6 and 9) in its interior. The opening 47 is configured to receive an advancing member 11 (see FIGS. 4A, 4B, 7, 8, 10, for example). The advancing member 11 may be part of or may be connected to the dosing mechanism of the device 1. The advancing member 11 is adapted and arranged to mechanically cooperate with the drive member 12 for rotating the drive member 12 in the first rotational direction. The advancing member 11 may comprise a drum. The advancing member 11 is rotatable. The advancing member 11 is rotatable around an axis which is parallel to the main longitudinal axis 16. The advancing member 11 is rotatable around the same axis around which the drive member 12 is rotatable. The advancing member 11 is rotatable around the third rotational axis 30. Preferably, the advancing member 11 is rotatable in the second rotational direction with respect to the drive member 12. The advancing member 11 may be prevented from rotation in the first rotational direction with respect to the drive member 12. In an alternative embodiment, the advancing member 11 is rotatable in the first rotation direction with respect to the drive member 12 and is prevented from rotation in the second rotational direction with respect to the drive member 12.

The advancing member 11 comprises an outer diameter. The outer diameter of the advancing member 11 is measured in a direction perpendicular to the axis around which the advancing member 11 is rotated, i.e. the third rotational axis 30. The outer diameter is smaller than an inner diameter of the drive member 12 such that the advancing member 11 can be inserted into the drive member 12 and, in particular, into the opening 47. In other words, the outer diameter of the advancing member 11 is smaller than the diameter of the opening 47.

The advancing member 11 is connected, preferably permanently connected, to the actuating element 54, in particular to the plunger member 39 (see, for example, FIGS. 3A and 3B). The plunger member 39 comprises the previously described connection member 17A, which is in the following referred to as the first connection member 17A. The advancing member 11 comprises a corresponding connection member 17B (see, for example FIGS. 7B and 14C), which is in the following referred to as the second connection member 17B. The connection members 17A, 17B are adapted and arranged to form a connection mechanism 17. The connection mechanism 17 may comprise a snap-fit mechanism. Due to mechanical cooperation of the connection members 17A, 17B, the plunger member 39 and the advancing member 11 are connected, in particular snap-fitted, to one another. The plunger member 39 and the advancing member 11 may be permanently connected to one another.

One of the connection members 17A, 17B may comprise at least one protrusion, e.g. two protrusions. The further connection member 17A, 17B may comprise at least one slot or indentation, for example. Preferably, the first connection member 17A comprises the protrusion. The first connection member 17A may comprise two protrusions. The second connection member 17B comprises the indentation. In other words, the plunger member 39 is at least partly inserted into the advancing member 11.

The first connection member 17A protrudes from the plunger member 39 in the proximal direction (see FIG. 2A). The first connection member 17A may also protrude from the plunger member 39 in the radial direction. The first connection member 17A comprises a proximal section 93 and a distal section 94. The proximal section comprises an extension perpendicular to the main longitudinal axis 16 which is greater than the extension perpendicular to the main longitudinal axis 16 of the distal section 94. The proximal section 93 may comprise a radial protrusion. The first connection member 17A may be shaped anchor-like, for example.

The second connection member 17B is designed as a recess within an interior of the advancing member 11 (see FIG. 7; the structure of the second connection member 17B applies for all embodiments). The second connection member 17B comprises a proximal section and a distal section. The proximal section comprises an extension perpendicular to the main longitudinal axis 16 which is greater than the extension perpendicular to the main longitudinal axis 16 of the distal section. In other words, the extension perpendicular to the main longitudinal axis 16 of the distal and proximal section of the second connection member 17B is adapted to the extension perpendicular to the main longitudinal axis 16 of the distal and proximal section of the first connection member 17A. An axial extension of the proximal section of the first connection member 17A may, however, be smaller than an axial extension of the proximal section of the second connection member 17B. This means that the first connection member 17A and, in particular its proximal section, may be axially slideable within the second connection member 17B, in particular in its proximal section. Further, the distal section of the first connection member 17A is slideably arranged with the distal section of the second connection member 17B.

The plunger member 39 and the advancing member 11 are, thus, slideably connected to one another. Before a suction airstream is created, i.e. before delivering a dose, the plunger member 39 is arranged with respect to the advancing member 11 in a first axial position, i.e. a proximal position. In the first axial or proximal position of the plunger member 39, the proximal section of the first connection member 17A abuts a proximal wall 50 of the proximal section of the second connection member 17B (see, for example, FIG. 7, whereby the proximal wall 50 is, of course, provided for both embodiments).

When a suction airstream is created for delivering a dose, the actuating element 54 and, thus, the plunger member 39 is—initially—axially moved in the distal direction with respect to the advancing member 11 towards the first position (see arrow 18 in FIGS. 7 and 14C). Thereby, the plunger member 39 is moved from the first axial position towards a second axial or distal position with respect to the advancing member 11. The actuating element 54 and, thus, the plunger member 39, is moved axially with respect to the advancing member 11 for a predetermined distance 48 until the plunger member 39 is arranged with respect to the advancing member 11 in the second axial position (see FIGS. 7 and 14C). The distance 48 is determined by the axial extension of the second connection member 17B and, in particular of the proximal section of the second connection member 17B. The second axial or distal position is arranged more distal than the first axial position. When the plunger member 39 is moved for the distance 48, the advancing member 11 is prevented from any movement with respect to the housing 3. In particular, the connection members 17A, 17B slide along each other such that movement of the plunger member 39 is prevented from being transferred to the advancing member 11.

When the plunger member 39 is arranged in the second axial position, the plunger member 39 and, in particular, the first connection member 17A mechanically cooperates with a distal wall, edge or protrusion 49 of the second interaction member 17B, in particular of its proximal section. When the plunger member 39 is arranged in the second axial position and upon further movement of the actuating element 54 in the distal direction towards the second position, the advancing member 11 is moved together with the plunger member 39 in the distal direction and at least partly out of the drive member 12. Thereby, the advancing member 11 is rotated (preferably in the second rotational direction) with respect to the drive member 12, which is described later on in detail.

When the cap 7 is mounted to the housing 3 after a dose delivery, the actuating element 54 is moved back towards the first position as described above. Thereby, the plunger member 39 is moved with respect to the advancing member 11 from the second axial position back towards the first axial position until the proximal section of the first connection member 17A abuts the proximal wall 50 of the second connection member 17B. Afterwards, the advancing member 11 is moved together with the plunger member 39 in the proximal direction (see arrow 26 in FIGS. 7 and 14D) until the actuating element 54 is positioned in the first position as described above.

When the advancing member 11 is moved proximally during re-engagement of the cap 7 and the housing 3, the advancing member 11 is prevented from any rotation. For this purpose, the plunger member 39 comprises a centering or alignment feature 21 (see FIGS. 7, 14B, 14D and 14E). The centering feature 21 comprises one, two or more prongs, for example. The centering feature 21 may comprise a toothing. The centering feature 21 and the plunger member 39 are integrally formed. The centering feature 21 protrudes from the plunger member 39 in the proximal direction. The centering feature 21 protrudes from the plunger member 39 in the radial direction.

When the plunger member 39 is moved towards the first axial position with respect to the advancing member 11, the centering feature 21 is brought into mechanical cooperation with the advancing member 11 and, in particular with an interaction feature 22 of the advancing member 11 (see, for example FIGS. 14E, 14F). The interaction feature 22 comprises a set of teeth. The interaction feature 22 projects or extends from the distal end section of the advancing member 11. The interaction feature 22 is arranged along the distal end section of the advancing member 11, for example.

When the plunger member 39 is arranged in the first axial position with respect to the advancing member 11 such that further axial movement between the advancing member 11 and the plunger member 39 is no longer possible during re-engagement of the cap 7, the centering feature 21 and the interaction feature 22 engage one another such that the advancing member 11 is prevented from any rotation with respect to the drive member 12 when the cap 7 is re-engaged and, in particular, when the advancing member 11 is moved in the proximal direction and towards the drive member 12.

When the actuating element 54 is moved further towards the first position, i.e. in the proximal direction, the advancing member 11 mechanically cooperates with the drive member 12 for rotating the drive member 12 in the first rotational direction, which is described now in detail.

The advancing member 11 comprises a first interaction member 28. The first interaction member 28 may comprise a set of teeth. A graduation of the first interaction member 28 may be greater than a graduation of the previously described interaction feature 22. In other words, the interaction feature 22 may comprise smaller teeth and more teeth than the interaction feature 28. A distance between two succeeding teeth of the interaction feature 22 (in particular a distance between the tips of the two succeeding teeth) may be smaller than a distance between two succeeding teeth of the first interaction member 28.

The first interaction member 28 projects or extends from the proximal end section of the advancing member 11. Alternatively, the first interaction member 28 may project radially from an outer surface of the advancing member, in particular a proximal end section of the advancing member 11. The first interaction member 28 is arranged along the proximal end section of the advancing member 11. The first interaction member 28 is directed in the proximal direction. The first interaction member 28 and, in particular one tooth of the set of teeth, comprises a first edge 28A and a second edge 28B. The first edge 28A is arranged along an axis parallel to the main longitudinal axis 16 of the device 1. The second edge 28B encloses an angle with the axis parallel to the main longitudinal axis 16. The first edge 28A is steeper than the second edge 28B.

The drive member 12 comprises a second interaction member 29. The second interaction member 29 comprises a set of teeth. These teeth are configured to mate with the teeth of the first interaction member 28. The second interaction 29 member projects or extends from an inner surface of the drive member 12. The second interaction member 29 projects into the opening 47. The second interaction member 29 projects in the distal direction. The second interaction member 29 and, in particular one tooth of the set of teeth, comprises a first edge 29A and a second edge 29B. The first edge 29A is arranged along an axis parallel to the third rotational axis 30. The second edge 29B encloses an angle with the axis parallel to the third rotational axis 30. The first edge 29A is steeper than the second edge 29B.

When the actuating element 54 is moved towards the first position, the advancing member 11 is moved proximally with respect to the drive member 12 due to mechanical cooperation with the plunger member 39 such that the first interaction member 28 is brought into mechanical cooperation with the second interaction member 29. Before the interaction members 28, 29 mechanically cooperate, the interaction members 28, 29 are arranged axially and/or angularly offset with respect to one another as it is obvious from FIGS. 8 and 14E. In other words, the interaction members 28, 28 are arranged in an axial and angular position with respect to one another such that the teeth of the first interaction member 28 are offset with respect to the teeth of the second interaction member 29. The angular offset may be smaller than or equal to 0.25 mm, for example. The axial offset may be smaller than or equal to 0.11 mm, for example. For further moving the advancing member 11 in the proximal direction, the interaction members 28, 29 must be brought in a meshed engagement.

Upon mechanical cooperation of the interaction members 28, 29, the first interaction member 28 slides along the second interaction member 29 (see, for example, FIG. 14E). In particular, the second edge 28B of the respective tooth of the first interaction member 28 slides down the second edge 29B of the respective tooth of the second interaction member 29. Thereby, the advancing member 11, in particular the first interaction member 28, exerts a force onto the drive member 12. Said force is directed in a direction perpendicular to the third rotational axis 30 (see arrow 27 in FIGS. 8 and 14F). The force is directed in the first rotational direction. Accordingly, the drive member 12 is rotated in the first rotational direction upon mechanical cooperation of the first and the second interaction members 28, 29. Thereby, the interaction members 28, 29 are brought in a meshed engagement. The advancing member 11 is prevented from rotation due to mechanical cooperation with the centering feature 21.

Upon rotation of the drive member 12, the display member 13 is further wound around the drive member 12 (first embodiment) or the rotating member 79 (second embodiment). The display member 13 is thereby moved for a predetermined distance which depends on the configuration of the interaction members 28, 29 (first embodiment) or on the configuration of the interaction members 28, 29 and the drive features 80, 81. The more oblique the second edge 28B, 29B with respect to the axis parallel to the main longitudinal axis 16, the greater may be the distance which the drive member 12 is rotated and, hence, which the display member 13 is moved. The greater the distance between succeeding teeth of the respective interaction member 28, 29 the greater may be the distance which the drive member 12 is rotated and, hence, which the display member 13 is moved.

The greater the distance between succeeding teeth of the respective driving feature 80, 81 the greater may be the distance which the rotating member 79 is rotated and, hence, which the display member 13 is moved. The teeth of the interaction members 28, 29 and/or the teeth of the respective driving feature 80, 81 may provide a mechanical advantage of the mechanism 5.

The mechanism may be adapted such that, when the drive member 12 is rotated about a first angle, the display member 13 is rotated about a second angle, wherein the second angle is different from the first angle. The second angle may be smaller than the first angle. For example, the first angle may amount to 90 degree and the second angle may be less than 90 degree, e.g. 60 degree, 50 degree, 45 degree, 30 degree or 15 degree. Alternatively, the first angle may amount to 45 degree or less and the second angle may be 30 degree, 20 degree, 15 degree or 10 degree.

Alternatively, the second angle may be greater than the first angle. For example, the second angle may amount to 90 degree and the first angle may be less than 90 degree, e.g. 60 degree, 50 degree, 45 degree, 30 degree or 15 degree. Alternatively, the second angle may amount to 45 degree or less and the first angle may be 30 degree, 20 degree, 15 degree or 10 degree.

In this way, a size of the indicia provided on the display member 13 may be chosen dependent on the mechanical advantage provided by the mechanism 5. Preferably, the indicia provided on the display member 13 comprise a height of smaller than or equal to 1.5 mm, e.g. 1.2 mm or 1.0 mm.

When the actuating element 54 is arranged in the first position, i.e. the most proximal position, the interaction member 28, 29 are in mating or meshing engagement. In other words, the respective tooth of the first engagement member 28 is arranged in a gap 51 in-between two adjacent teeth of the second interaction member 29. Now, further movement of the advancing member 11 in the proximal direction with respect to the drive member 12 is no longer possible. Thus, the advancing member 11 is arranged in a proximal end position with respect to the drive member 12.

When the cap 7 is removed again from the device 1 and a further dose of the substance 2 is delivered, the plunger member 39 is moved distally with respect to the advancing member 11 such that the centering feature 21 and the interaction feature 22 disengage. Accordingly, the advancing member 11 is rotatable with respect to the drive member 12.

Afterwards, the plunger member 39 and the advancing member 11 move together in the distal direction with respect to the drive member 12 as described above due to their mechanical cooperation. However, for enabling further rotation of the drive member 12 in the first rotational direction, the advancing member 11 must be rotated such that, when the cap 7 is re-engaged, the interaction member 28, 29 comprise a predetermined axial and/or angular orientation with respect to one another. In particular, the advancing member 11 must be rotated such that, when the cap 7 is re-engaged, the first interaction member 28 is arranged in a position with respect to the second interaction member 29 such that the first interaction member 28 can slide along the second interaction member 29 as described above. In other words, the interaction members 28, 29 must be arranged offset with respect to one another when the cap 7 is re-engaged. Otherwise, the respective tooth of the first interaction member 28 would be received in the respective gap 51 without exerting a force onto the drive member 12.

The advancing member 11 is rotated, preferably rotated in the second rotational direction, upon delivering a dose of the substance 2, for example. However, the advancing member 11 is prevented from rotation in the first rotational direction when delivering a dose. For this purpose, the advancing member 11 may comprise a projection feature 90 (see FIG. 4A), e.g. a toothing. The projection feature 90 may be arranged on an outer surface, in particular around the outer surface, of the advancing member 11. The device 1 may comprise an engagement feature, e.g. a pawl (not explicitly shown in the Figures). Mechanical cooperation of the pawl and the projection feature 90 may prevent rotation of the advancing member 11 in the first rotational direction during dose delivery, i.e. when the advancing member 11 is moved towards the mouthpiece 6.

For rotating the advancing member 11 in the second rotational direction, the advancing member comprises the previously described projection feature or toothing 90. The dosing mechanism and, in particular the actuator housing 35, comprises a reset feature 23. The reset feature 23 may protrude from the actuator housing 35 and through a hole or opening in the actuating element 54. The reset feature 23 and the actuator housing 35 are integrally formed. The reset feature 23 comprises at least one protrusion or prong. Alternatively, the reset feature 23 may comprise a set of teeth. The reset feature 23 projects or extends from the actuator housing 35. The reset feature 23 projects from the actuator housing 35 in a proximal direction and towards the projection feature or toothing 90.

The reset feature 23 is adapted and arranged to mechanically cooperate with the toothing 90 when the advancing member 11 is moved in the distal direction during dose delivery. The reset feature 23 and the toothing 90 are configured such that they are permanently arranged offset with respect to one another. In other words, a tip of a respective tooth of the toothing 90 is arranged angularly offset with respect to a tip of the reset feature 23, in particular of the prong, of the protrusion or of a tooth of the reset feature 23. Preferably, a tip of a respective tooth of the toothing 90 is arranged 0.1 mm or 0.2 mm angulary offset from the tip of the prong, the protrusion or one respective tooth of the reset feature 23. In this way, the reset feature 23 and the toothing 90 are always arranged offset with respect to one another such that the reset feature 23 can exert a force onto the toothing 90 for rotating the advancing member 11.

When the reset feature 23 and the toothing 90 start to mechanically cooperate upon movement of the advancing member 11 in the distal direction, the reset feature 23 and the toothing 90 slide along each other. Thereby, the reset feature 23 exerts a force onto the advancing member 11 such that the advancing member 11 is rotated in the second rotational direction with respect to the drive member 12. However, the reset feature 23 and the toothing 90 are not brought in alignment, in particular meshed engagement, with one another due to rotation of the advancing member 11. Rather, as described above, the reset feature 23 and the toothing 90 are still arranged offset with respect to one another after rotation of the advancing member 11 due to the specific configuration and arrangement of the reset feature 23 and the toothing 90, in particular due to a specific and small graduation of the toothing 90. In particular, the graduation of the toothing 90 may be smaller than the graduation of the first interaction member 28 (see FIG. 4A).

Due to rotation of the advancing member 11, the first interaction member 28 is brought in a position offset from the second interaction member 29 as described above for rotating the second interaction member 29 in the first rotational direction when the advancing member 11 is moved proximally during re-engagement of the cap 7 and the housing 3. When the advancing member 11 is moved proximally during re-engagement of the cap 7 and the housing 3, the reset feature 23 and the toothing 90 disengage and the advancing member 11, in particular the interaction feature 22 is brought into engagement with the centering feature 21 such that any further rotation of the advancing member 11 is prevented as described above.

In the following, the assembly of the mechanism 5 and the device 1 is described. Most of the assembly steps apply for the mechanism 5 of both the first and the second embodiment.

A) The rotating member 79 is put on the body 8′. In particular, the rotating member 79 is put on the mounting element 82 such that the rotating member 79 is rotatable in the first rotational direction but such that it is prevented from rotation in the second rotational direction. Step A) applies only for the second embodiment. In the first embodiment, the rotating member 79 is redundant. Thus, the method for assembling the mechanism 5 according to the first embodiment starts with step B).

B) The drive member 12 is put on the body 8, 8′. In particular, the drive member 12 is put on the mounting element 91 such that the drive member 12 is rotatable in the first rotational direction but such that it is prevented from rotation in the second rotational direction. This step applies for the first and the second embodiment. When assembling the drive member 12 onto the body 8′, the drive member 21 and, in particular the drive feature 80, may be brought into engagement with the rotating member 79 and, in particular with the drive feature 81 (this applies only for the second embodiment).

C) The advancing member 11 is inserted into the drive member 12. The advancing member 11 is inserted without orientation of the interaction members 28, 29 with respect to one another. This step applies for the first and the second embodiment.

D) The rotatable member 32 is put on the body 8, 8′. In particular, the rotatable member 32 is put on the mounting element 83 such that the rotatable member 32 is rotatable in the first rotational direction (first embodiment) or, alternatively, in the first and second rotational direction (second embodiment). Alternatively, the rotatable member 32 may be rotatable in the first and in the second rotational direction also in the first embodiment. The display member 13, in particular the first end of the display member 13, is attached to the rotatable member 32. The display member 13 is almost completely wound around the rotatable member 32. This step applies for both the first and the second embodiment.

E) The display member 13, in particular the second end of the display member 13, is attached to the drive member 12 (first embodiment). In particular, in the first embodiment, a respective pair of drive features 19 is brought into engagement with a respective pair of interaction members 20. The display member 13 is attached to the drive member 12 such that it is at least partly wound around the drive member 12. The display member 13, in particular a portion of the display member 13, may be led along the support member 84. The display member 13 is lead along the support member 84 such that there is direct mechanical contact with the drive member 12 (see FIG. 5).

Alternatively, (second embodiment), the display member 13, in particular the second end of the display member 13, is attached to the rotating member 79. Thereby, the display member 13, in particular a portion of the display member 13 is led along the support member 84 such that the display member 13 passes along the drive member 12 without having direct mechanical contact with the drive member 12. The display member 13 is attached to the rotating member 79 such that it is at least partly wound around the rotating member 79.

F) The rotatable member 32 may be turned forwards (in the first rotational direction) and backwards (in the second rotational direction) such that the display member 13 is stretched. Accordingly, the display member 13 is clamped between the rotatable member 32 and the rotating member 79 is a stretched condition. This step may apply only for the second embodiment. Alternatively, the rotatable member 32 may be rotatable in the first and in the second rotational direction also in the first embodiment. In this case, the rotatable member 32 may be turned forwards (in the first rotational direction) and backwards (in the second rotational direction) such that the display member 13 is clamped between the rotatable member 32 and the drive member 12 in a stretched condition.

G) The mechanism 5 is now ready to be inserted into the actuator housing 35. In particular, the mounting elements 9, 10 are brought into engagement for snap fitting the body 8, 8′ to the actuator housing 35 (see FIG. 2G and FIGS. 2E, 2F, 3A, 3B). The mechanism 5 is assembled to the actuator housing 35 from the side as can be seen in FIG. 2G.

H) The actuating element 54 is at least partly inserted into the actuator housing 35. The actuating element 54 is inserted such that the plunger 39 is lead through the opening 88 (see FIGS. 2C and 2D). Thereby, the plunger 39 and, in particular the first connection member 17A, is brought into engagement with the advancing member 11, in particular with the second connection member 17B.

I) The further components of the device 1 are assembled for achieving the device shown in FIGS. 1A, 1B and 1C.

Other implementations are within the scope of the following claims. Elements of different implementations may be combined to form implementations not specifically described herein.

1-14. (canceled)

15. An assembly for a counter mechanism for a drug delivery device comprising
a drive member, wherein the drive member is adapted and arranged to be rotated in a first rotational direction and to be prevented from rotation in a second rotational direction which direction is opposite to the first rotational direction,
an advancing member adapted and arranged to be axially moved and rotated with respect to the drive member, wherein the advancing member is adapted and arranged to mechanically cooperate with the drive member such that the drive member is rotated in the first rotational direction,
a display member which is configured to count a number of doses and to display the counted number of doses,
wherein the assembly is adapted and arranged such that rotation of the drive member in the first rotational direction is converted into a counting movement of the display member,
wherein the advancing member comprises a first interaction member and the drive member comprises a second interaction member,
wherein the interaction members are configured to mechanically cooperate with one another when they are arranged offset with respect to one another in a predetermined axial and/or angular orientation and when the advancing member is moved in the first axial direction such that the first interaction member slides along the second interaction member, thereby exerting a force onto the drive member for rotating the drive member in the first rotational direction, and
wherein the interaction members are configured to mechanically cooperate with one another when they are not arranged offset with respect to one another in the axial and/or angular orientation and when the advancing member is moved in the first axial direction such that the first interaction member is received in the second interaction member, without exerting a force onto the drive member.

16. The assembly according to claim 15, wherein the advancing member is configured to mechanically cooperate with the drive member when the advancing member is moved in a first axial direction with respect to the drive member, and wherein the advancing member is prevented from mechanical cooperation with the drive member when the advancing member is moved in a second axial direction with respect to the drive member, wherein the second axial direction is opposite to the first axial direction.

17. The assembly according to claim 15, further comprising a plunger member which is moveable during at least one of a dose dispensing operation and a dose setting operation, wherein the plunger member is configured to mechanically cooperate with the advancing member such that the advancing member is axially moveable with respect to the drive member, wherein the plunger member comprises a first connection member and the advancing member comprises a second connection member, wherein the connection members are configured to mechanically cooperate with one another such that the plunger member is slideably connected to the advancing member.

18. The assembly according to claim 17, wherein the plunger member is axially moveable with respect to the advancing member between a first axial position and a second axial position, wherein,
(i) when the plunger member is arranged in the first axial position, movement of the plunger member in the first axial direction is converted into movement of the advancing member in the first axial direction due to mechanical cooperation of the connection members,
(ii) when the plunger member is arranged in the second axial position, movement of the plunger member in the second axial direction is converted into movement of the advancing member in the second axial direction due to mechanical cooperation of the connection members, and
(iii) when the plunger member is moved between the first axial position and the second axial position, the connection members are in a sliding engagement such that movement of the advancing member with respect to the drive member is prevented.

19. The assembly according to claim 15, wherein the interaction members are arranged offset with respect to one another before the interaction members mechanically cooperate when the advancing member is moved towards the drive member.

20. The assembly according to claim 15, wherein the first interaction member comprises a set of teeth and wherein the second interaction member comprises a set of mating teeth, and wherein the interaction members are in a meshed engagement when the advancing member is positioned in an end position with respect to the drive member after the advancing member was moved towards the drive member.

21. The assembly according to claim 16, further comprising a centering feature, wherein the advancing member comprises an interaction feature adapted and arranged to prevent rotation of the advancing member with respect to the drive member when the advancing member is moved in the first axial direction due to mechanical cooperation with the centering feature, and wherein the interaction feature comprises a set of teeth.

22. The assembly according to claim 16, further comprising a reset feature, wherein the advancing member comprises a projection feature, wherein the reset feature and the projection feature are configured to mechanically cooperate with one another when the advancing member is moved in the second axial direction such that the reset feature and the projection feature slide along each other, the reset feature thereby exerting a force onto the advancing member for rotating the advancing member with respect to the drive member.

23. The assembly according to claim 15, wherein the display member and the drive member are in direct mechanical contact with one another such that rotation of the drive member in the first rotational direction is converted into the counting movement of the display member.

24. The assembly according to claim 15, further comprising a rotating member which is coupled with the display member, wherein the rotating member and the drive member are configured to mechanically cooperate with one another such that rotation of the drive member in the first rotational direction is converted into the counting movement of the display member.

25. The assembly according to claim 24, wherein the drive member comprises a first driving feature and the rotating member comprises a second driving feature and wherein the driving features are configured to engage with one another for transferring movement of the drive member in the first rotational direction into movement of the rotating member in the first rotational direction.

26. The assembly according to claim 15, wherein the assembly is adapted such that, when the drive member is rotated about a first angle, the display member is rotated about a second angle, wherein the second angle is different from the first angle.

27. The assembly according to claim 15, wherein the assembly is adapted such that in plan view onto the display member, the display member covers the drive member at least partly.

28. A drug delivery device comprising the assembly according to claim 15.
